SEQUENCE LISTING (11 sequences)

SEQ ID NO: 1 - DNA, 987 bp, Homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 2 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 99 aa, Homo sapiens [sequence omitted]

SEQ ID NO: 4 - protein, 99 aa, Homo sapiens
IQRTPKIQVYSRHPAENGKSNFLNCYVSGFHPSDIEVDLLKNGERIEKVEHSDLSFSKDWSFYLLYYTEFTPTEKNEYACRVNHVTLSQPKIVKWDRDM

SEQ ID NO: 5 - DNA, 32 bp, Artificial Sequence
  Description of Artificial Sequence Synthetic primer
cacccccactgaaaaaaatgagtatgcctgcc

SEQ ID NO: 6 - DNA, 24 bp, Artificial Sequence
  Description of Artificial Sequence Synthetic primer
catatgatccagcgtactccaaag

SEQ ID NO: 7 - DNA, 28 bp, Artificial Sequence
  Description of Artificial Sequence Synthetic primer
gccggatccttacatgtctcgatcccac

SEQ ID NO: 8 - DNA, 18 bp, Homo sapiens
  CDS: (2)..(16)
tgaaaaagatgagtatgc

SEQ ID NO: 9 - protein, 5 aa, Homo sapiens
EKDEY

SEQ ID NO: 10 - protein, 6 aa, Homo sapiens
DEYACR

SEQ ID NO: 11 - protein, 6 aa, Homo sapiens
NEYACR

(19) United States
(12) Patent Application Publication
(10) Pub. No.: US 2013/0331327 A1
(43) Pub. Date: Dec. 12, 2013

(54) Variant Beta2-microglobulin, characterization of the same and applications thereof

(71) Applicants: Vittorio Bellotti, Pavia (IT); Sophie Valleix, Saint-Maur-des-Fosses (FR); Mark Brian Pepys, London (GB); Madeleine M. Riès-Kautt, Paris (FR)

(72) Inventors: Vittorio Bellotti, Pavia (IT); Sophie Valleix, Saint-Maur-des-Fosses (FR); Mark Brian Pepys, London (GB); Madeleine M. Riès-Kautt, Paris (FR)

(21) Appl. No.: 13/507,159

(22) Filed: Jun. 8, 2012

(51) Int. Cl. C07K 14/435; C12N 15/63; C12N 1/21; A61P 43/00; G01N 33/68; C12N 5/10; C12N 1/19; A61K 38/17; C12N 15/12; C07K 1/02

(52) U.S. Cl. 514/13.5; 530/394; 536/23.5; 435/320.1; 435/252.33; 436/86; 435/348; 435/254.2; 435/325

(57) ABSTRACT

This invention relates to the specific Asp76Asn (D76N) variant β₂-microglobulin (β₂M) protein, nucleic acids encoding the same, their characterization and applications in studying amyloid fibrillogenesis, including diagnostic and therapeutic applications.

### INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

This patent application contains a lengthy table. Copies of TABLE 1 have been submitted in duplicate in compact disc (CD) form as finalized CD-R discs (i.e., “Copy 1” and “Copy 2”) in accordance with MPEP 608.05 and are hereby incorporated herein by reference, and may be employed in the practice of the invention. Each compact disc, created Jun. 8, 2012 contains the following file: Table_{—}1_D76N_def.PDB, 167 KB.

### FIELD OF THE INVENTION

This invention relates to an isolated or recombinant or engineered specific Asp76Asn (D76N) variant β₂-microglobulin (β₂M) protein, nucleic acids encoding the same, their characterization, recombinant plasmids, vectors or host cells expressing them, methods of generating amyloid fibrils composed of variant β₂M protein in vitro under essentially physiological conditions, use of this protein to study amyloid fibrillogenesis, diagnostic and therapeutic applications of the variant β₂M protein to screen for modulators of amyloid fibril formation, therapeutic compositions of inhibitors of amyloid fibril formation and disruptors of amyloid fibrils identified in said screens and methods of treatment using said therapeutic compositions.

### BACKGROUND OF THE INVENTION

In humans, amyloid is a pathological extracellular deposit composed predominantly of characteristic abnormal protein fibrils associated with proteoglycans and glycosaminoglycans of heparin and dermatan types, and the non-fibrillar pentraxin protein, serum amyloid P component (SAP). Amyloid deposits are the direct cause of tissue damage and disease in the condition known as amyloidosis, which may be systemic, affecting the viscera, connective tissues and blood vessel walls throughout the body, or local, when the amyloid deposits are confined to just one anatomical area or organ system. Amyloidosis is a major unmet medical need because both the local and the systemic forms cause serious morbidity, the diagnosis is usually made very late when there is already irreversible organ damage, and both amyloidotic tissue damage and the acquired and hereditary conditions which are responsible for amyloid deposition are often very intractable. Furthermore there are as yet no treatments which specifically target the amyloid deposits themselves. Systemic amyloidosis is almost universally fatal and causes about one per thousand of all deaths in developed countries.

Amyloid deposits are also always present in Alzheimer's disease and type 2 diabetes but the clinical significance and pathogenic role of the amyloid is not known, and these conditions should thus not be considered as forms of amyloidosis at this time. Similarly Parkinson's disease and Huntington's disease are associated with abnormal protein aggregates but these are intracellular and it is both wrong and misleading to conflate them with amyloid and amyloidosis.

The genuine cerebral amyloid deposits which are present in some, but not all, forms of the spongiform encephalopathies caused by prions, are clearly not necessary for disease to be manifest and these conditions are thus also not forms of amyloidosis.

About 30 different proteins are known to form amyloid fibrils in vivo in humans but regardless of the particular amyloid fibril protein, all amyloid deposits have an amorphous eosinophilic appearance in hematoxylin and eosin stained histological sections viewed by light microscopy and they stain specifically with alkaline alcoholic Congo red and then give pathognomonic red/green birefringence when viewed under cross polarized light, preferably strong cross polarized light (Puchtler, H., Sweat, F. and Levine, M. (1962) On the binding of Congo red by amyloid. J. Histochem. Cytochem., 10: 355-364). Amyloid deposits all have a fibrillar ultrastructure when viewed by transmission electron microscopy. Amyloid fibrils of all types, when isolated from tissues, are rigid, non-branching structures of indeterminate length with diameter 8-12 nm and composed of twisted protofibrils. All amyloid fibrils share a common pathognomonic cross-β sheet core structure (Sunde, M., Serpell, L. C., Bartlam, M., Fraser, P. E., Pepys, M. B., Blake, C. C.F. Common core structure of amyloid fibrils by synchrotron X-ray diffraction. J. Mol. Biol. 1997; 273: 729-739). Amyloid deposits form in vivo when there is: (i) sustained exposure to either normal or increased concentrations of a normal, potentially amyloidogenic, protein; (ii) when an abnormal amyloidogenic protein is produced as a consequence of an acquired disease; or (iii) when a gene mutation encodes an amyloidogenic variant protein (Pepys, M. B. (2006) Amyloidosis. Annu. Rev. Med., 57: 223-241). Amyloid fibrillogenesis results from reduced stability of the native fold of the fibril precursor protein, so that it populates partly unfolded intermediate states which aggregate as stable amyloid fibrils with the characteristic cross-β core structure (Booth, D. R., Sunde, M., Bellotti, V., Robinson, C. V., Hutchinson, W. L., Fraser, P. E., Hawkins, P. N., Dobson, C. M., Radford, S. E., Blake, C. C. F. and Pepys, M. B. (1997) Instability, unfolding and aggregation of human lysozyme variants underlying amyloid fibrillogenesis. Nature, 385: 787-793). Almost any protein can form amyloid-like fibrils in vitro if treated harshly enough but in vivo amyloid fibrillogenesis only occurs with precursors which are sufficiently unstable to misfold and then aggregate under physiological conditions.

The nomenclature of amyloidosis, established by the nomenclature committee of the International Society for Amyloidosis (Sipe J D, Benson M D, Buxbaum J N, Ikeda S, Merlini G, Saraiva M J, Westermark P. Amyloid fibril protein nomenclature: 2010 recommendations from the nomenclature committee of the International Society of Amyloidosis. Amyloid. 2010;17, 101-4.), is based on the protein which forms the amyloid fibrils in each type of the disease. All types have the prefix A, representing amyloid, followed by an abbreviation for the amyloid fibril protein. The main forms of systemic amyloidosis are (i) AL type in which the fibrils are composed of monoclonal immunoglobulin light chains produced by B cell or plasma cell clones in the various forms of plasma cell dyscrasia, including multiple myeloma and non-invasive monoclonal gammopathy, (ii) AA type with fibrils composed of amyloid A protein derived from the acute phase reactant, serum amyloid A protein (SAA), which is produced in enormously increased amounts in all acute and chronic inflammatory conditions, (iii) Aβ₂M with fibrils composed of β₂-microglobulin (β₂M) which accumulates to very high concentration in the plasma of individuals with end stage renal failure who are on dialysis, so-called dialysis related amyloidosis (DRA), and (iv) ATTR with fibrils composed of wild type transthyretin (TTR) which occurs with increasing frequency in old age and predominantly affects the heart causing senile cardiac amyloidosis. There are also many different forms of hereditary systemic amyloidosis, caused by mutations in a number of different plasma proteins. Although the condition is very rare indeed, affecting probably not more than about 12,000 people worldwide, it is also very important because these late onset autosomal dominant diseases continue to be transmitted in families, are almost always fatal and there is little or no effective prophylaxis or treatment for most types. Hereditary amyloidosis is also of great importance because identification of the amyloid fibril proteins and the precise structural and functional effects of the amyloidogenic mutations have enabled the molecular mechanisms of amyloid fibrillogenesis to be elucidated in considerable molecular detail (Booth, D. R., Sunde, M., Bellotti, V., Robinson, C. V., Hutchinson, W. L., Fraser, P. E., Hawkins, P. N., Dobson, C. M., Radford, S. E., Blake, C. C. F. and Pepys, M. B. (1997) Instability, unfolding and aggregation of human lysozyme variants underlying amyloid fibrillogenesis. Nature, 385: 787-793; Hammarström P, Schneider F, Kelly J W. Trans-suppression of misfolding in an amyloid disease. Science. 2001;293: 2459-62). This is notably the case for the variant form of human β₂M which is the subject of the present application and which opens the way to both improved understanding of amyloid fibrillogenesis and also the development of new therapeutic interventions.

Current treatment for systemic amyloidosis aims to reduce the abundance of the amyloid fibril protein precursor, since this is known to arrest new amyloid accumulation and, in some patients, to allow regression of existing amyloid deposits, all of which is associated with reduced morbidity and prolonged survival. Crucially, patients must survive long enough for the various treatments aimed at the fibril precursor proteins to be effective, and this requires comprehensive, extensive, expert medical management up to and including organ function support and transplantation, sometimes of several organs. Unfortunately many of the treatments required to reduce fibril protein precursor production are very expensive, toxic, poorly tolerated and slow to act. Combined with the fact that amyloidosis is rare, and thus unfamiliar to most physicians, it is difficult to diagnose. The clinical manifestations are protean and the diagnosis must be thought of, tissue biopsy must be performed and sophisticated specific histological and/or imaging procedures undertaken. AL amyloidosis is by far the most common form of systemic amyloidosis and although quality of life and survival have improved greatly for many patients over the past 30 years, about 20% of individuals with AL disease still die within 12 months of diagnosis. Diagnosis, treatment and outcomes of all types of amyloidosis are much better in expert specialist centres but there is huge scope for improvement, especially in developing treatments which specifically target amyloid fibril formation and elimination rather than just precursor protein abundance. The uniquely highly amyloidogenic variant form of human β₂M which is the subject of this application will play a pivotal role in design, discovery and development of drugs targeting all types of amyloidosis.

Human β₂-microglobulin (β₂M), molecular weight 11,815 Da, is an invariant, non-polymorphic cell surface protein present on all nucleated cells throughout the body. It is the constant chain of the highly polymorphic Major Histocompatibility Complex (MHC) class I molecules which are crucial for self non-self recognition by the immune system. β₂M is non-covalently associated with the polymorphic MHC class I a-chain and in adult humans about 200 mg per day of free β₂M is shed into the plasma. Due to its small molecular size, free β₂M is swiftly and completely filtered in the renal glomerulus and this is the only route for its disposal from the body. β₂M is specifically taken up from the glomerular filtrate by the cells of the proximal convoluted renal tubule and catabolised therein. This very efficient process ensures that circulating plasma concentrations of β₂M are in a tight range around 1-2 mg/I in normal healthy subjects. With reduced glomerular filtration rate and loss of renal tubules in renal failure, the plasma β₂M concentration rises and in end stage renal failure requiring dialysis, the concentration reaches 50 mg/1 or more. Furthermore neither peritoneal dialysis nor routine hemodialysis removes more than a minor proportion of β₂M from the blood, so that the circulating concentration remains persistently around 50-70 mg/l. After 5-7 years on dialysis, nearly all such patients develop deposits of β₂M amyloid, mostly in and around bones and joints although rare cases with significant visceral amyloid deposits have also been described. The β₂M amyloid deposits cause a range of different, painful and often crippling clinical effects including carpal tunnel syndrome, serious large joint arthropathy, ligament rupture, and bone cysts leading to pathological fractures. Until recently dialysis related amyloidosis (DRA) was universal in all patients who had been dialysed for 10 years or more and constituted the major long term morbidity associated with long term dialysis. The recent introduction of high flux membranes, which allow for transport of much more β₂M and are also composed of more biocompatible materials, has been associated with reduced incidence of DRA. However with about 1 million individuals on long term dialysis worldwide, DRA remains a major unmet medical need.

There has accordingly been intense research effort aimed at understanding the mechanisms of β₂M amyloid fibrillogenesis in vitro and in vivo, including its remarkable localisation specifically to bones and joints and notable sparing of the viscera, unlike any other form of systemic amyloidosis. β₂M has always been a tempting molecule to study because of its small size and already fully solved 3D structure when it was first recognised as an amyloid fibril protein. However intact native wild type β₂M is stable under physiological conditions and correspondingly poorly amyloidogenic. In order to form amyloid fibrils in vitro, low pH and/or denaturing solvents, and long incubations are required, although the process can be markedly accelerated and enhanced by seeding with preformed amyloid fibrils and/or extracellular components such as glycosaminoglycans and SAP (Myers S L et al. A systematic study of the effect of physiological factors on beta-2-microglobulin amyloid formation at neutral pH). Much of the β₂M extracted from β₂M amyloid deposits ex-vivo has undergone cleavage of the N-terminal 6 amino acid residues and this truncated form of β₂M is much more fibrillogenic than the full length protein. It also binds much more avidly to collagen in vitro, suggesting that cleavage of intact β₂M may be an important aspect of the pathogenesis of DRA. Other suggestions about mechanisms of β₂M amyloid fibrillogenesis have been based on site directed mutagenesis and expression of recombinant forms of β₂M with enhanced propensity to aggregate (Eichner T, Radford S E. Understanding the complex mechanisms of beta2-microglobulin amyloid assembly. Febs J 2011; 278:3868-83).

In marked contrast, and completely unpredicted by any prior art, the β₂M variant which is the subject of the present application contains a single amino acid substitution in a previously unexplored and totally unexpected region of the protein. Despite the substitution enabling an additional new hydrogen bond to form in the native structure, the variant protein is nonetheless thermodynamically destabilised and spontaneously aggressively forms typical amyloid fibrils from solutions at physiological pH and ionic strength without any destabilising solvents. These properties make it an ideal model system for exploration of the molecular mechanisms of amyloid fibrillogenesis and for design, discovery, testing and development of novel therapeutics aimed at stabilising the native fold of amyloidogenic proteins, inhibiting fibrillogenic aggregation and promoting disruption of formed fibrils. Active compounds identified in this way will be of use in prevention and treatment of DRA and also, given the stereotyped core structure and assembly of all amyloid fibrils, for applications in many or even all other forms of amyloidosis and amyloid associated diseases.

Citation or identification of any document in this application is not an admission that such document is available as prior art to the present invention.

### SUMMARY OF THE INVENTION

The present invention provides, in different embodiments, the novel amino acid sequence of a particular, isolated or recombinant or engineered specific variant β₂M protein containing the D76N substitution and the nucleic acid sequence encoding the same, which may optionally be used as reagents to study amyloid fibril formation.

An embodiment of the invention provides recombinant plasmids, vectors or host cells expressing the variant β₂M protein.

An embodiment of the invention provides a composition comprising variant β₂M protein that is amyloidogenic under essentially physiological conditions in vitro.

A further embodiment of the invention is a method wherein amyloid fibrils comprising variant β₂M protein are generated in vitro which have similar characteristics to amyloid fibrils associated with β₂M amyloidosis in humans in vivo.

An embodiment of the invention is to provide a high throughput drug screening system wherein inhibitors of variant β₂M amyloid fibril formation are identified.

An embodiment of the invention is to provide a high throughput drug screening system wherein the effects of compounds available from libraries on aggregates and/or amyloid fibrils comprising variant β₂M protein are studied.

A further embodiment of the invention provides therapeutic compositions of inhibitors of amyloid fibril formation and/or disruptors of amyloid fibrils identified in said screens and methods of treatment using said therapeutic compositions.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1A shows a family tree indicating the occurrence of β₂M amyloid in a French family. The different symbols in the family tree indicate that the β₂M mutation is present; that there is a clinical syndrome; that β₂M amyloid is confirmed histologically; that SAP scintigraphy is positive for amyloid; and that there is no clinical syndrome or β₂M mutation.

FIG. 1B shows microscopic images in which abundant hepatic amyloid is seen around portal vessels (arrows) in patient II.1 (top left: Congo red stain, bright field original magnification×200; top middle: indirect immunofluorescence with anti-β₂M antibody; original magnification×200; top right: no significant staining was observed when the anti-β₂M antibody was omitted; original magnification×200). Vascular amyloid in colonic submucosa in patient II.9 (lower left: indirect immunofluorescence with anti-β₂M antibody; original magnification×200; lower middle: no staining was observed when the anti-β₂M antibody was omitted; original magnification×200). Cardiac amyloid fibrils in patient II.1 (lower right: electron microscopic immunogold staining with anti-β₂M antibody; original magnification×80,000).

FIG. 1C shows the partial sequence chromatogram of the β₂M gene showing the nucleotide substitution identified in the four affected patients (II.1, II.7, II.9, and II.2).

FIG. 1D shows posterior views of planar whole body ¹²³I-labeled SAP scans from patients II.7 (left) and II.9 (middle) showing amyloid in the spleen and adrenal glands of both cases. A posterior whole body ¹²³I-labeled SAP scan from a subject without amyloidosis is shown (right) for comparison.

FIG. 2A shows a negatively stained transmission electron micrograph of amyloid fibrils extracted from the spleen of patient II.1; scale bar, 100 nm.

FIG. 2B shows a graph depicting size exclusion chromatography of amyloid fibrils solubilized in 20 mM sodium phosphate pH 7.4 containing 6 M guanidine hydrochloride. The inset within the figure shows a western blot of fractions, numbered on the chromatogram, immunostained with polyclonal anti β₂M antibody.

FIG. 2C shows graphs depicting matrix assisted laser desorption/ionization time of flight spectrometric analysis of the trypsin digest of the SDS-PAGE separated band from fraction 4 of the chromatogram in FIG. 2B. The tryptic peptide 76-81, mass 812.37, derived from the ex vivo fibrils is identical to the peptide obtained from digestion of recombinant D76N β₂M, mass 812.30, and both are distinct from the tryptic peptide of recombinant wild type β₂M, mass 813.27, which is 1 Da larger. Furthermore, the mass of the peptide containing residue Asn76 demonstrated that the residue was not glycosylated, consistent with the absence from the protein of the typical consensus sequence for N-glycosylation.

FIG. 2D shows a graph depicting equilibrium guanidine hydrochloride denaturation curves of wild type β₂M (blue, circles) and D76N variant β₂M (red, squares) monitored at pH 7.4 and 30° C. by intrinsic fluorescence emission. Data were fitted using a two state unfolding model (Santoro M M, Bolen D W. Unfolding free energy changes determined by the linear extrapolation method. 1. Unfolding of phenylmethanesulfonyl alpha-chymotrypsin using different denaturants. Biochemistry 1988;27:8063-8) and shown as fraction of unfolded protein Fapp=(y−yN)/(yU−yN), where y is the emission at 330 nm, yN and yU are the same values for the native and unfolded proteins.

FIG. 2E shows a graph depicting the kinetics of fibril formation monitored by thioflavin T binding assay. Wild type β₂M (blue, circles) and D76N variant β₂M (red, squares) were each incubated at 40 μM in 25 mM phosphate buffer pH 7.4 containing 100 μg/ml heparin and 25 μg/ml wild type β₂M fibril seeds at 37° C. with agitation at 225 rpm. The variant aggregated in the same time frame even in absence of heparin (black, triangles).

FIG. 2F shows a negatively stained transmission electron micrograph of D76N variant β₂M amyloid-like fibrils formed in vitro in 25 mM phosphate buffer pH 7.4 containing 100 μg/ml heparin and 25 μg/ml wild type β₂M fibril seeds; scale bar, 100 nm.

FIG. 3A shows the ribbon representation of the D76N variant with the N76 residue indicated within the square in the image.

FIG. 3B shows a close up of the EF loop (residues 70-80) superposed onto the wild type β₂M structure. Residues T73, N76 and Y78 belonging to the D76N variant are indicated; Tyr78 belonging to wild type β₂M is also indicated; H-bonds are shown as dashed lines.

FIG. 3C shows an image depicting the surface electrostatic potential of the EF loop region in the D76N variant (above) and in wild type β₂M (below).

### Sequences

SEQ ID NO: 1 shows a wild type β₂M mRNA molecule of accession number NM_{—}004048.
SEQ ID NO: 2 shows a wild type β₂M gene translation product including a 20 amino acid signal peptide.
SEQ ID NO: 3 shows a mature wild type β₂M protein transcript excluding signal peptide.
SEQ ID NO: 4 shows a mature mutant or variant β₂M protein transcript excluding signal peptide.
SEQ ID NO: 5 shows a QuickChange™ site-directed mutagenesis kit and primer sequence CACCCCCACTGAAAAAAATGAGTATGCCTGCC used for mutagenesis of Asp76 into Asn.

### Tables

TABLE 1 (filed on a CD): The crystallographic coordinates of the Asp76Asn (D76N) variant β₂-microglobulin (β₂M) protein.

### DETAILED DESCRIPTION

β₂M has a mass of 11,815 Da and is the invariant light chain of the human HLA class I complex. It is cleared only via the kidney, at ˜200 mg/day in adults, so that its plasma concentration rises from the normal 1-2 mg/L to ˜50 70 mg/L in patients with end stage renal failure on dialysis (Floege J, Bartsch A, Schulze M, Shaldon S, Koch K M, Smeby L C. Clearance and synthesis rates of beta 2-microglobulin in patients undergoing hemodialysis and in normal subjects. J Lab Clin Med 1991;118:153-65). This results in DRA which is a serious and intractable condition, with β₂M amyloid fibrils deposited in bones and joints, causing painful arthropathy, cysts and pathological fractures, and there may also be rare visceral β₂M amyloid deposits (Gejyo F, Yamada T, Odani S, et al. A new form of amyloid protein associated with chronic hemodialysis was identified as beta 2-microglobulin. Biochem Biophys Res Commun 1985;129:701-6). The normal structure and function of β₂M are well characterized and fibrillogenesis of β₂M is widely studied. Although the wild type protein is poorly amyloidogenic in vitro, many recombinant β₂M variants have been investigated and hypotheses developed about mechanisms of fibrillogenesis (Eichner T, Radford S E. Understanding the complex mechanisms of beta2-microglobulin amyloid assembly. Febs J 2011;278:3868-83).

The present invention pertains to the novel identification and isolation of variant β₂M proteins and the nucleic acids encoding the same. The variant β₂M protein of the invention is the first pathologically relevant, amyloidogenic variant of β₂M. It was discovered in members of a French family who developed progressive bowel dysfunction with extensive visceral amyloid deposits composed of β₂M. In contrast to DRA, the patients all had normal circulating concentrations of β₂M and normal renal function. The pathogenic protein is aggressively fibrillogenic in vitro, prompting re-evaluation of previously hypothesized mechanisms of β₂M fibrillogenesis.

The invention relates to the very first report of autosomal dominant hereditary β₂M amyloidosis. The clinical phenotype comprises onset in middle age of altered bowel habit, caused by both direct gastrointestinal amyloid deposition and autonomic neuropathy, accompanied by sicca syndrome, and has a very slowly progressive clinical course culminating in extensive, widespread, visceral amyloid deposition. Each of the four affected individuals in the kindred was heterozygous for a mutation encoding D76N variant β₂M, and their amyloid deposits contained only the full length variant protein. The clinical manifestations differ from those caused by the selective bone and joint related wild type β₂M amyloid deposits in DRA, which is a complication of the sustained gross elevation of the circulating β₂M concentration in patients with end stage renal failure (Munoz-Gomez J, Bergoda-Barado E, Gomez-Perez R, et al. Amyloid arthropathy in patients undergoing periodical haemodialysis for chronic renal failure: a new complication. Ann Rheum Dis 1985;44:729-33.) The formation of pathogenic β₂M amyloid deposits in vivo despite normal plasma concentrations of β₂M presumably reflects the aggressive fibrillogenicity of the D76N variant under physiological conditions. The late disease onset and slowly progressive clinical course are consistent with its low plasma concentration. The absence of osteoarticular amyloid deposits is consistent with the low affinity of β₂M for collagen (Giorgetti S, Rossi A, Mangione P, et al. Beta2-microglobulin isoforms display an heterogeneous affinity for type I collagen. Protein Sci 2005;14:696-702). Significant adsorption of β₂M onto collagen only occurs at the very high β₂M concentrations attained during haemodialysis. Also the truncated form, ΔN6-β₂M, which is ubiquitous in dialysis related amyloid deposits but absent in the present kindred, binds to collagen with much higher affinity than does full length β₂M (Giorgetti S, Rossi A, Mangione P, et al. Beta2-microglobulin isoforms display an heterogeneous affinity for type I collagen. Protein Sci 2005;14:696-702).The organ tropism may thus be dictated by β₂M truncation, as has been hypothesized for other amyloid proteins (Pepys MB. A molecular correlate of clinicopathology in transthyretin amyloidosis. J Pathol 2009;217:1-3). The ratio of circulating variant to wild type β₂M, which has previously been reported to be less than 1:1 for other amyloidogenic proteins (Mangione P, Sunde M, Giorgetti S, et al. Amyloid fibrils derived from the apolipoprotein A1 Leu174Ser variant contain elements of ordered helical structure. Protein Sci 2001;10:187-99), is currently under investigation but is a non-trivial undertaking given the low β₂M concentration and the very limited availability of patient plasma. It is noteworthy that, despite the misfolding propensity of the D76N β₂M variant and the essential contribution of β₂M to the structure of the HLA class I complex, none of the heterozygotes showed clinical evidence of immune deficiency.

The new hydrogen-bonds, Asn76-Tyr78 and Tyr78-Thr73, appear to stabilize the residue 73-78 region of the polypeptide sequence that connects the E and F strands of the protein (EF loop). Indeed, the high B-factors of the EF loop, 165% of the molecule average, indicate that this is the most flexible part of the structure of wild type β₂M, whereas the equivalent region of the D76N variant is more rigid with reduced B-factors, 120% of average. The molecular mechanisms of the instability and amyloidogenicity are intriguing since they are manifest despite the greater rigidity of native protein folding inferred from the crystal structure. Other amyloidogenic variant globular proteins, Va130Met transthyretin (Hamilton J A, Steinrauf L K, Braden B C, et al. The X-ray crystal structure refinements of normal human transthyretin and the amyloidogenic Val-30→Met variant to 1.7-Å resolution. J Biol Chem 1993;268:2416-24) and Thr57Ile lysozyme (Booth D R, Sunde M, Bellotti V, et al. Instability, unfolding and aggregation of human lysozyme variants underlying amyloid fibrillogenesis. Nature 1997;385:787-93) also have essentially wild type crystal structures and yet very abnormal folding dynamics, though these are not as marked as for the D76N β₂M variant. The observations are consistent with the idea that amyloidogenic proteins are capable of making fibrils whilst maintaining a native fold (Chiti F, Dobson C M. Amyloid formation by globular proteins under native conditions. Nat Chem Biol 2009;5:15-22) and suggest that only limited conformational modification may be sufficient to promote fibrillogenesis.

The fibrillogenicity of D76N β₂M under essentially physiological conditions enables testing, in a clinically relevant milieu, of potential therapeutic inhibitors. Some of the small molecule ligands that have been recently identified as inhibitors of wild type β₂M fibrillogenesis (Giorgetti S, Raimondi S, Pagano K, et al. Effect of tetracyclines on the dynamics of formation and destructuration of beta2-microglobulin amyloid fibrils. J Biol Chem 2011;286:2121-31; Domanska K, Vanderhaegen S, Srinivasan V, et al. Atomic structure of a nanobody-trapped domain-swapped dimer of an amyloidogenic beta2-microglobulin variant. Proc Natl Acad Sci U S A 2011;108:1314-9), have been found to be excellent inhibitors of D76N β₂M fibrillogenesis in preliminary studies.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T.

Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual : Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988,

Cold Spring Harbor Laboratory Press, ISBN 0-87969-314-2), 1855, Lars-Inge Larsson “Immunocytochemistry: Theory and Practice”, CRC Press inc., Baca Raton, Florida, 1988, ISBN 0-8493-6078-1, John D. Pound (ed); “Immunochemical Protocols, vol 80”, in the series: “Methods in Molecular Biology”, Humana Press, Totowa, New Jersey, 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

In describing the various embodiments of the invention, a number of terms are defined for clarity. Terms not defined should be accorded their ordinary meanings as used in the relevant art.

As used herein, “wild-type”, “natural” or “wild-type natural” genes or proteins are those found in nature. The terms “wild-type sequence” and “wild-type gene/protein” are used interchangeably herein, to refer to a sequence that is naturally occurring in a host cell.

As used herein, the term “isolated” or “non-naturally occurring” refers to sequences that are at least substantially free from at least one other component with which the sequence is naturally associated in nature and as found in nature. Similarly the terms “engineered” or “recombinant” all indicate the involvement of the hand of man. In one aspect, preferably the sequence is in a purified form. The term “purified” means that the sequence is in a relatively pure state—e.g. at least about 90% pure, or at least about 95% pure or at least about 98% pure.

As used herein, the terms “polypeptide” and “protein” are used interchangeably to refer to polymers of any length comprising amino acid residues linked by peptide bonds. The conventional one-letter or three-letter code for amino acid residues is used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins”. Such proteins may be derived from organisms of different genera and/or species, or even different classes of organisms (e.g., primate, mouse, drosophila, bacteria etc.). Related proteins also encompass homologs determined by primary sequence analysis, determined by secondary or tertiary structure analysis, or determined by immunological cross-reactivity.

As used herein, the term “derivative polypeptide/protein” refers to a protein which is derived from a protein by addition of one or more amino acids to either or both the N- and C-terminal end(s), substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. The preparation of a protein derivative may be achieved by modifying a DNA sequence which encodes the protein, transformation of that DNA sequence into a suitable host, and expression of the modified DNA sequence to form the derivative protein.

As used herein, “variant” shall mean a molecule being derivable from a parent molecule. Variants shall include polypeptides as well as nucleic acids. Variants shall include substitutions, insertions, transversions and inversions, among other things, at one or more locations. Variants shall also include truncations. Variants shall include homologous and functional derivatives of parent molecules. Related (and derivative) proteins include “variant proteins”. Variant proteins differ from a reference/parent protein, e.g., a wild-type protein, by substitutions, deletions, and/or insertions at small number of amino acid residues. The number of differing amino acid residues may be one or more, for example, 1, 2, 3, 4, 5, 6, 7, 8, 9, 10, 15, 20, 30, 40, 50, or more amino acid residues. Variant proteins are proteins having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, identity or amino acid sequence identity with a reference protein. A variant protein may also differ from a reference protein in single amino acids, selected motifs, domains, epitopes, conserved regions, and the like.

As used herein, the term “analogous sequence” refers to a sequence within a protein that provides similar function, tertiary structure, and/or conserved residues as the protein of the invention. For example, in epitope regions that contain an alpha-helix or a beta-sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some embodiments, the tertiary structure and/or conserved residues of the amino acids in the protein of interest are located at or near the segment or fragment of interest. Thus, where the segment or fragment of interest contains, for example, an alpha-helix or a beta-sheet structure, the replacement amino acids preferably maintain that specific structure.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences may be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, WI); and Devereux et al. (1984) Nucleic Acids Res. 12:387-395).

For example, PILEUP is a useful program to determine sequence homology levels.

PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Dooittle, (Feng and Doolittle (1987) J. Mol. Evol. 35:351-360). The method is similar to that described by Higgins and Sharp (Higgins and Sharp (1989) CABIOS 5:151-153).

Useful PILEUP parameters including a default gap weight of 3.00, a default gap length weight of 0.10, and weighted end gaps. Another example of a useful algorithm is the BLAST algorithm, described by Altschul et al. (Altschul et al. (1990) J. Mol. Biol. 215:403-410; and Karlin et al. (1993) Proc. Natl. Acad. Sci. USA 90:5873-5787). One particularly useful BLAST program is the WU-BLAST-2 program (See, Altschul et al. (1996) Meth. Enzymol. 266:460-480). Parameters “W,” “T,” and “X” determine the sensitivity and speed of the alignment. The BLAST program uses as defaults a word-length (W) of 11, the BLOSUM62 scoring matrix (See, Henikoff and Henikoff (1989) Proc. Natl. Acad. Sci. USA 89:10915) alignments (B) of 50, expectation (E) of 10, M′S, N′-4, and a comparison of both strands.

As used herein, the phrases “substantially similar” and “substantially identical”, in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity may be determined using known programs such as BLAST, ALIGN, and CLUSTAL using standard parameters. (See, e.g., Altschul, et al. (1990) J. Mol. Biol. 215:403-410; Henikoff et al. (1989) Proc. Natl. Acad. Sci. USA 89:10915; Karin et al. (1993) Proc. Natl. Acad. Sci USA 90:5873; and Higgins et al. (1988) Gene 73:237-244). Software for performing BLAST analyses is publicly available through the

National Center for Biotechnology Information. Also, databases may be searched using FASTA (Pearson et al. (1988) Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “deletion of a gene”, refers to its removal from the genome of a host cell.

As used herein, “disruption of a gene” refers broadly to any genetic or chemical manipulation that substantially prevents expression of a function gene product, e.g., a protein, in a host cell. Exemplary methods of disruption include complete or partial deletion of any portion of a gene (including a polypeptide-coding sequence, a promoter, an enhancer, or another regulatory element, or mutagenesis of the same (including substitutions, insertions, deletions, and combinations, thereof), to substantially prevent expression of a function gene product.

As used herein, “a functional gene” is a gene capable of being used by cellular components to produce an active gene product, typically a protein. “Functional” genes are the antithesis of “disrupted” genes, which are modified such that they cannot be used by cellular components to produce an active gene product.

An embodiment of the invention provides purified variant β₂M nucleic acids and proteins generated by recombinant means.

A further embodiment of the invention provides recombinant plasmids, vectors or host cells comprising variant β₂M nucleic acid sequences and expressing the variant β₂M protein. As used herein, “recombinant” is used to describe molecules that result from the use of laboratory methods (molecular cloning) to bring together genetic material from multiple sources, creating sequences that would not otherwise be found in biological organisms.

The nucleic acids of the invention can be prepared according to standard recombinant DNA techniques. A nucleotide sequence encoding the variant β₂M protein can be determined using the genetic code and an oligonucleotide molecule having this nucleotide sequence can be synthesized by standard DNA synthesis methods (e.g., using an automated DNA synthesizer). Alternatively, a DNA molecule encoding the variant β₂M protein can be derived from the natural or wild-type β₂M precursor protein gene or cDNA (e.g., using the polymerase chain reaction and/or restriction enzyme digestion) according to standard molecular biology techniques.

Accordingly, the invention further provides an isolated nucleic acid molecule comprising a nucleotide sequence encoding the variant β₂M protein, such that more than one nucleic acid molecule sequence may be arrived at taking into account the redundancy of the genetic code.

As used herein, the term “nucleic acid molecule” is intended to include DNA molecules and RNA molecules and may be single-stranded or double-stranded.

To facilitate expression of a variant protein in a host cell by standard recombinant DNA techniques, the isolated nucleic acid encoding the protein is incorporated into a recombinant expression vector. Accordingly, the invention also provides recombinant expression vectors comprising the nucleic acid molecules of the invention. As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors, which serve equivalent functions.

In the recombinant expression vectors of the invention, the nucleotide sequence encoding the variant protein are operatively linked to one or more regulatory sequences, selected on the basis of the host cells to be used for expression. The term “operably linked” is intended to mean that the sequences encoding the peptide compound are linked to the regulatory sequence(s) in a manner that allows for expression of the peptide compound. The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cell, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of peptide compound desired, etc. The expression vectors of the invention can be introduced into host cells thereby to produce peptide compounds encoded by nucleic acids as described herein.

The recombinant expression vectors used in methods of the invention can be designed for expression of variant proteins in prokaryotic or eukaryotic cells. For example, expression may be in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector may be transcribed and translated in vitro, for example using T7 promoter regulatory sequences and T7 polymerase. Examples of vectors for expression in yeast S. cerivisae include pYepSecl (Baldari et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of proteins or peptides in cultured insect cells (e.g., Sf 9 cells) include the pAc series (Smith et al., (1983) Mol. Cell. Biol. 3:2156-2165) and the pVL series (Lucklow, V. A., and Summers, M. D., (1989) Virology 170:31-39). Examples of mammalian expression vectors include pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40.

In addition to the regulatory control sequences discussed above, the recombinant expression vector may contain additional nucleotide sequences. For example, the recombinant expression vector may encode a selectable marker gene to identify host cells that have incorporated the vector. Such selectable marker genes are well known in the art. Moreover, the facilitate secretion of the peptide compound from a host cell, in particular mammalian host cells, the recombinant expression vector preferably encodes a signal sequence operatively linked to sequences encoding the amino-terminus of the peptide compound such that upon expression, the peptide compound is synthesized with the signal sequence fused to its amino terminus. This signal sequence directs the peptide compound into the secretory pathway of the cell and is then cleaved, allowing for release of the mature peptide compound (i.e., the peptide compound without the signal sequence) from the host cell. Use of a signal sequence to facilitate secretion of proteins or peptides from mammalian host cells is well known in the art.

A recombinant expression vector comprising a nucleic acid encoding the β₂M variant protein can be introduced into a host cell to thereby produce the variant protein in the host cell. Accordingly, the invention also provides host cells containing the recombinant expression vectors of the invention. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell may be any prokaryotic or eukaryotic cell. For example, a peptide compound may be expressed in bacterial cells such as E. coli, insect cells, yeast or mammalian cells. Preferably, the variant protein is expressed in mammalian cells.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those that confer resistance to drugs, such as G418, hygromycin and methotrexate. Nucleic acid encoding a selectable marker may be introduced into a host cell on the same vector as that encoding the peptide compound or may be introduced on a separate vector. Cells stably transfected with the introduced nucleic acid can be identified by drug selection (e.g., cells that have incorporated the selectable marker gene will survive, while the other cells die).

Methods utilized to express and purify the wild type and variant β₂M proteins may be as described in Esposito et al. Removal of the N-terminal hexapeptide from human beta2-microglobulin facilitates protein aggregation and fibril formation. Protein Sci. 2000 May;9(5):831-45; Rennella et al. Equilibrium Unfolding Thermodynamics of β2-Microglobulin Analyzed through Native-State H/D Exchange. Biophys J. 2009 January 7; 96(1): 169-179 and Esposito G, Ricagno S, Corazza A, et al. The controlling roles of Trp60 and Trp95 in beta2-microglobulin function, folding and amyloid aggregation properties. J Mol Biol 2008;378:887-97, the disclosures of all of which are incorporated by reference herein. As an example the β2M cDNA may be obtained in a pHN1 plasmid. The expression cassette for the full-length β2M was created by using PCR with β2M cDNA as the template and the oligonucleotides primers 5′CATATGATCCAGCGTACTCCAAAG and 3′GCCGGATCCTTACATGTCTCGATCCCAC for the full-length and containing the underlined NdeI restriction sites at the 5′ and the BamHI restriction site at the 3′. The amplification mixture (40 μL) contained dGTP, dATP, dTTP, dCTP (each 200 μM), oligonucleotide primers (1 μM), template DNA (10 ng), Thermus aquaticus (Taq) DNA polymerase (Pharmacia, Uppsala, Sweden) in 40 mM NaCl, 10 mM MgCl₂ 50 mM Tris-HC1, pH 9. The reaction mixture were subjected to 2 cycles of 94° C. for 1 min, 45° C. for 1 min, 72° C. for 1 min and 30 cycles in which the annealing was obtained at 50° C. A final incubation at 72° C. for 10 min ended the cycles. The amplified DNA was gel purified, digested with BamHI and NdeI, and ligated into pet 11a vector that has been digested with the same enzymes. The Escherichia coli strain BL21DE3 was transformed with the plasmids and clones containing inserts and producing protein upon induction with isopropyl β-_{D} thiogalactopyranoside (IPTG) were identified. The correct nucleotide sequence of the insert was confirmed by DNA sequencing. A methionine residue was always present at the N-terminal position of the recombinant products and is referred to as M0 and is usually not taken into account with regards to amino acid positions in the sequence. The variant β₂M protein may be expressed using the same steps. Protein reconstitution and purification may involve the following methodology. One liter of cells was transformed with the full-length β₂M and was incubated at 37° C. and induced to express the protein by addition of 1 mM IPTG. The cells were harvested by centrifugation and the cell pellets containing the recombinant β₂M as inclusion bodies were resuspended in 10 mM Tris HCl, pH 8.0 (20 mL) containing lysozyme at 100 μg/mL and phenylmethylsulfonyl fluoride (PMSF) at 50 μg/mL. The cells were lysed by sonication and then centrifuged (10,000×g) for 20 min. The pellet containing recombinant protein was washed with 10 mM Tris HC1, pH 8.0 (20 mL), dissolved in 100 mM Tris HC1, pH 8.0/6 M GdnHC1 (10 mL), and centrifuged (100,000 ×g) for 1 h at 4° C. The supernatant was applied to a Sephacryl S300 column equilibrated with the 6 M GdnHC1 buffer. The identification of chromatographic fractions containing β₂M was accomplished by ELISA. β₂M isolated from gel filtration was then dialyzed against 10 mM Tris HCl, pH 8.0 and further purified by ion exchange chromatography with an UNO column (Bio-Rad, Hercules, Calif.). Electrospray mass spectrometry and DNA sequencing may be used to confirm the identity of the recombinant and variant proteins and compared with wild-type β₂M.

An embodiment of the invention provides a composition comprising variant β₂M protein that is amyloidogenic under essentially physiological conditions in vitro. The term “in vitro” is well known in the art and generally refers to conditions created within environments which include but are not limited to a test tube, plastic reaction tubes or microcentrifuge tubes, cell culture flask, petri dish or multi-well plates, and includes a wide range of cell and tissue culture techniques, along with cell-free methods.

The term “essentially physiological conditions” as used herein are those conditions which are present in the normal human extracellular fluid environment. They include the physiological pH, salt concentration, total ionic strength and buffer capacity. The term “essentially physiological conditions” does not require conditions identical to those in the extracellular environment of living humans, where β₂M exists in the body, but conditions which are closely similar. The person skilled in the art will, of course, realize that certain constraints may arise due to the experimental set-up which will eventually lead to less physiological conditions. For example, if an embodiment of the invention requires the utilization of any type of cells and extraction of the gene or protein from that cell, the eventually necessary disruption of cell walls or cell membranes when taking and processing the gene or protein may require conditions which are not identical to the physiological conditions found in the organism. Suitable variations of physiological conditions for practicing the methods of the invention will be apparent to those skilled in the art and are encompassed by the term “essentially physiological conditions” as used herein. It is to be understood that the term “essentially physiological conditions” relates to conditions close to physiological conditions, as e.g. found in natural extracellular environments, such as the plasma or extracellular tissue fluid in different tissues and organs, but does not necessarily require that these conditions are identical.

For example, “essentially physiological conditions” with regards to the fibrillogenesis of D76N variant β₂M is carried out in conditions mimicking the composition and physical properties of the extracellular tissue fluid. This contrasts sharply with amyloid fibrillogenesis by all other known amyloidogenic globular proteins. These require adverse, non-physiological conditions, such as very low pH and sometimes also the presence of denaturing or chaotropic solvents, and/or seeding with preformed fibrils and/or other additives. Uniquely the D76N variant β₂M which is the subject of the present application forms fibrils under conditions closely related to those prevailing in vivo. Thus the physiological ionic strength is close to 150 mM and provided mostly by NaCl, with buffering by sodium phosphate at 15-50 mM and the pH is within the range 6.5-7.5 in order to mimic different possible states of acidosis. The colloidal component is provided by inclusion of isolated human or bovine serum albumin within the concentration range of 1-3% w/v. Glycosaminoglycans, such as heparin at 30-50 μg/ml, slightly accelerate the rate of fibrillogenesis but are not essential for the formation of amyloid fibrils. The kinetics of formation of fibrils is highly dependent on the presence and intensity of shear force applied to the protein solution and the simultaneous presence of hydrophobic surfaces, such as air at the water-air interface or the presence of hydrophobic collagen or carbon nanotubes. A shear force of at least 50 pN at the interface of the hydrophobic and hydrophilic surfaces is required. Embodiments of the invention may relate to a temperature range of 2-40° C., preferably 4-37° C.

With regards to fibril formation under essentially physiological conditions, in some embodiments of the invention heparin may be added to the solution. In other embodiments, heparin may not be added. As used herein the term “fibril seeds” refers to small aggregates of fibrils that “seed” elongated fibril assembly. In some embodiments of the invention, fibril seeds may be present to propagate fibril formation and in other embodiments of the invention, fibril seeds may not be used to initiate fibril formation or amyloid formation. If heparin is added, preferably 30-50 μg/ml of heparin is added; if fibril seeds are present they are preferably present at a concentration of 25 μg/ml. Methods of agitation or agitation means may or may not be used in other embodiments of the invention. These may include but are not limited to magnetic stirrers, mechanical shakers, shaking incubators and centrifuges. If agitation or an agitation means is used in an embodiment of the invention, agitation is preferably about 175- 275 rpm, more preferably about 200-250 rpm and most preferably 225 rpm. The amount of polypeptide used to generate amyloid fibrils or study amyloid formation under essentially physiological condition may be about 20-60 μM, preferably about 30-50 μM and most preferably about 40 μM.

As used herein, the term “saline” or “saline solution” refers to a solution of NaCl in water or essentially a salt water solution. The solution may be sterile when used parenterally such as in intravenous administration. Saline solutions are available in various formulations in which the concentration of NaCl varies from low to high. Normal Saline is defined as a solution of 0.90% w/v NaCl.

An embodiment of the invention provides a composition comprising the variant (β₂M protein and further comprising an aggregation enhancing agent which may include but is not limited to cations which include but are not limited to Cu²⁺, Mg²⁺ and Ca²⁺; glycosaminoglycans which include but are not limited chondroitin sulfate, dermatan sulfate, keratin sulfate, heparin, heparin sulfate and hyaluronan; lysophosphatidic acid; non-esterified fatty acids and collagen. Aggregation enhancing agents are described in more detail in Eakin C M, Miranker A D. From chance to frequent encounters: origins of beta2-microglobulin fibrillogenesis. Biochim Biophys Acta. 2005;1753:92-99; Srikanth R, Mendoza V L, Bridgewater J D, Zhang G, Vachet R W. Copper binding to beta2-microglobulin and its pre-amyloid oligomers. Biochemistry. 2009;48:9871-9881; Calabrese M F, Miranker A D. Metal binding sheds light on mechanisms of amyloid assembly. Prion. 2009;3:1-4; Blaho D V, Miranker A D. Delineating the conformational elements responsible for Cu(2+)-induced oligomerization of beta2-microglobulin. Biochemistry. 2009;48:6610-6617; Calabrese M F, Eakin C M, Wang J M, Miranker A D. A regulatable switch mediates self-association in an immunoglobulin fold. Nat Struct Mol Biol. 2008;15:965-971; Antwi K, Mahar M, Srikanth R, Olbris M R, Tyson J F, Vachet RW. Cu(II) organizes beta2-microglobulin oligomers but is released upon amyloid formation. Protein Sci. 2008;17:748-759; Calabrese M F, Miranker A D. Formation of a stable oligomer of beta2-microglobulin requires only transient encounter with Cu(II) J Mol Biol. 2007;367:1-7; Eakin C M, Berman A J, Miranker A D. A native to amyloidogenic transition regulated by a backbone trigger. Nat Struct Mol Biol. 2006;13:202-208; Deng N J, Yan L, Singh D, Cieplak P. Molecular basis for the Cu2+ binding-induced destabilization of beta2-microglobulin revealed by molecular dynamics simulation. Biophys J. 2006;90:3865-3879; Pal-Gabor H, Gombos L, Micsonai A, Kovacs E, Petrik E, Kovacs J, Graf L, Fidy J, Naiki H, Goto Y, et al. Mechanism of lysophosphatidic acid-induced amyloid fibril formation of beta2-microglobulin in vitro under physiological conditions. Biochemistry. 2009;48:5689-5699; Relini A, Canale C, De Stefano S, Rolandi R, Giorgetti S, Stoppini M, Rossi A, Fogolari F, Corazza A, Esposito G, et al. Collagen plays an active role in the aggregation of beta2-microglobulin under physiopathological conditions of dialysis-related amyloidosis. J Biol Chem. 2006;281:16521-16529; Relini A, De Stefano S, Torrassa S, Cavalleri O, Rolandi R, Gliozzi A, Giorgetti S, Raimondi S, Marchese L, Verga L, et al. Heparin strongly enhances the formation of beta2-microglobulin amyloid fibrils in the presence of type I collagen. J Biol Chem. 2008;283:4912-4920 and Giorgetti S, Rossi A, Mangione P, Raimondi S, Marini S, Stoppini M, Corazza A, Viglino P, Esposito G, Cetta G, et al. Beta2-microglobulin isoforms display an heterogeneous affinity for type I collagen. Protein Sci. 2005;14:696-702, the disclosures of all of which are incorporated by reference herein.

A further embodiment of the invention is a method wherein amyloid fibrils comprising variant β₂M protein are generated in vitro under essentially physiological conditions which have similar characteristics to amyloid fibrils associated with β₂M amyloidosis in humans in vivo. Another embodiment of the invention is a method wherein amyloid fibrils comprising variant β₂M protein are generated in vitro under essentially physiological conditions under a controlled shear rate(Y=94.2/sec) and with air exposure of at least 5% of the solvent surface acting as a further aggregation enhancing agent. In the absence of air the fibrillogenesis of D76N β₂M can be converted into amyloid-like fibrils within a few hours in physiologic buffer by a combination of a shear rate of 90/sec and the presence of hydrophobic surfaces, such as carbon nanotubes, occupying 0.5% of the fluid or solution volume.

Another embodiment of the invention is a method to screen for and identify modulators, i.e. enhancers or inhibitors, of variant β₂M gene activity. The screens may be conducted using other eukaryotic systems and libraries as several approaches have been used to map gene interactions. It may be determined if the gene encoding the human variant β₂M protein or a closely related homologous protein interacts with genes in other eukaryotic systems and if an interaction is identified then the relevant homologous human gene may be subsequently identified and further experiments to elucidate the interaction may be carried out. Genetic interaction screens based on synergy and antagonism have been performed on a large scale using synthetic genetic array analysis in yeast (Tong A H, Evangelista M, Parsons A B, Xu H, Bader G D, Page N, Robinson M, Raghibizadeh S, Hogue C W, Bussey H, et al. 2001. Systematic genetic analysis with ordered arrays of yeast deletion mutants. Science 294: 2364-2368; Tong A H, Lesage G, Bader G D, Ding H, Xu H, Xin X, Young J, Berriz G F, Brost R L, Chang M, et al. 2004. Global mapping of the yeast genetic interaction network. Science 303: 808-813; Costanzo M, Baryshnikova A, Bellay J, Kim Y, Spear E D, Sevier C S, Ding H, Koh J L, Toufighi K, Mostafavi S, et al. 2010. The genetic landscape of a cell. Science 327: 425-431) and RNA interference in Caenorhabditis elegans (Lehner B, Crombie C, Tischler J, Fortunato A, Fraser A G, 2006. Systematic mapping of genetic interactions in Caenorhabditis elegans identifies common modifiers of diverse signaling pathways. Nat Genet 38: 896-903.), covering about 30% and about 0.03% of all potential interactions, respectively.

Synthetic Genetic Array analysis (SGA) is a high-throughput technique that allows one to explore synthetic lethal and synthetic sick genetic interactions (SSL). With SGA a systematic construction of double mutants using a combination of recombinant genetic techniques, mating and selection steps can be achieved. Using SGA methodology a query gene deletion mutant can be crossed to an entire genome deletion set to identify any SSL interactions to generate functional information of the query gene and the genes it interacts with. Results obtained from such a study tend to show that genes with similar function tend to interact with one another and genes with similar patterns of genetic interactions often encode products that tend to work in the same pathway or complex. SGA was initially developed using the model organism S. cerevisiae and has been also used in S. pombe and E. coli. SGA is generally conducted using colony arrays on petriplates at standard densities (such as 96, 384, 768, 1536). To perform a SGA analysis in S. cerevisae, the query gene deletion is crossed systematically with a deletion mutant array (DMA) containing every viable knockout ORF of the yeast genome. The resulting diploids are then sporulated by transferring to a media containing reduced nitrogen. The haploid progeny are then put through a series of selection platings and incubations to select for double mutants. The double mutants are screened for SSL interactions visually or using imaging software by assessing the size of the resulting colonies. A similar methodology may be used to get functional information on the variant β₂M protein or related homologous proteins.

RNA interference (RNAi) is a process that moderates the activity of genes within living cells. Two types of small ribonucleic acid (RNA) molecules—microRNA (miRNA) and small interfering RNA (siRNA)—play key roles in RNAi. These small RNAs can bind to other specific mRNA molecules and either increase or decrease their activity, for example by preventing an mRNA from producing a protein. RNAi has an important role in defending cells against parasitic genes as well as in directing development as well as gene expression in general. Genome-scale RNAi research relies on the high-throughput screening technology which is described further in a subsequent section. This technology when combined with RNAi allows genome-wide loss-of-function screening and is broadly used in the identification of genes associated with specific biological phenotypes and allows one to simultaneously interrogate thousands of genes. The basic process of cell-based RNAi screening includes (i) choosing an RNAi library, (ii) selecting a robust and stable type of cells, (iii) transfecting the selected cells with RNAi agents from the chosen RNAi library, (iv) performing any necessary treatment or incubation steps, (v) detecting a signal, (vi) performing statistical and bioinformatics analysis, and (vii) determining which genes are important genes or therapeutical targets.

Protein-protein interactions have been assessed using yeast two-hybrid mapping or co-affinity immunoprecipitation (Cusick M E, Klitgord N, Vidal M, Hill D E. 2005. Interactome: Gateway into systems biology. Hum Mol Genet 14 (Spec no. 2): R171-R181; Rual J F, Venkatesan K, Hao T, Hirozane-Kishikawa T, Dricot A, Li N, Berriz G F, Gibbons F D, Dreze M, Ayivi-Guedehoussou N, et al. 2005. Towards a proteome-scale map of the human protein-protein interaction network. Nature 437: 1173-1178) in yeast, C. elegans, and Drosophila melanogaster (Giot L, Bader J S, Brouwer C, Chaudhuri A, Kuang B, Li Y, Hao Y L, Ooi C E, Godwin B, Vitols E, et al. 2003. A protein interaction map of Drosophila melanogaster. Science 302: 1727-1736; Li S, Armstrong C M, Bertin N, Ge H, Milstein S, Boxem M, Vidalain P O, Han J D, Chesneau A, Hao T, et al. 2004. A map of the interactome network of the metazoan C. elegans. Science 303: 540-543; Gavin A C, Aloy P, Grandi P, Krause R, Boesche M, Marzioch M, Rau C, Jensen L J, Bastuck S, Dumpelfeld B, et al. 2006. Proteome survey reveals modularity of the yeast cell machinery. Nature 440: 631-636; Yu H, Braun P, Yildirim M A, Lemmens I, Venkatesan K, Sahalie J, Hirozane-Kishikawa T, Gebreab F, Li N, Simonis N, et al. 2008. High-quality binary protein interaction map of the yeast interactome network. Science 322: 104-110 ; Simonis N, Rual J F, Carvunis A R, Tasan M, Lemmens I, Hirozane-Kishikawa T, Hao T, Sahalie J M, Venkatesan K, Gebreab F, et al. 2009. Empirically controlled mapping of the Caenorhabditis elegans protein-protein interactome network. Nat Methods 6: 47-54.). The proportion of potential protein-protein interactions evaluated is more than 77% in yeast, about 50% in Drosophila, and about 25% in C. elegans.

Two-hybrid mapping or screening is a molecular biology technique used to discover protein-protein interactions and protein-DNA interactions by testing for physical interactions (such as binding) between two proteins or a single protein and a DNA molecule, respectively. The general concept behind the test is the activation of downstream reporter gene(s) by the binding of a transcription factor onto an upstream activating sequence (UAS). In two-hybrid screening, the transcription factor is split into two separate fragments, called the binding domain (BD) and activating domain (AD). The BD is the domain responsible for binding to the UAS and the AD is the domain responsible for the activation of transcription. This method of screening or mapping is thus a protein-fragment complementation assay. Two-hybrid mapping often utilizes a genetically engineered strain of yeast in which the biosynthesis of certain nutrients (usually amino acids or nucleic acids) is lacking. When the yeast is grown on media that lacks these nutrients, they do not survive. This mutant yeast strain can be made to incorporate foreign DNA in the form of plasmids and in two-hybrid screening, separate bait and prey plasmids are simultaneously introduced into the mutant yeast strain. Plasmids are engineered to produce a protein product in which the DNA-binding domain (BD) fragment is fused onto a protein while another plasmid is engineered to produce a protein product in which the activation domain (AD) fragment is fused onto another protein. The protein fused to the BD is referred to as the bait protein, and is typically the protein that is known and the one that is used to identify new binding partners. The prey protein is the protein fused to the AD and can be either a single known protein or a library of known or unknown proteins. Libraries are constructed to consist of a collection of protein-encoding sequences that represent all the proteins expressed in a particular organism or tissue, or may also be generated by synthesizing random DNA sequences. Subsequently, these proteins are incorporated into the protein-encoding sequence of a plasmid, which is then transfected into the cells selected for the screening method. If the bait and prey proteins interact (i.e., bind), then the AD and BD of the transcription factor are connected in an indirect manner, bringing the AD close to the transcription start site and transcription of reporter gene(s) can thus occur. If the two proteins do not interact, the reporter gene is not transcribed. To particularly address human or mammalian protein-protein interactions, one may construct a two hybrid cDNA library that covers the entire human genome. A homologous recombination-mediated approach may be used to construct a modular human EST-derived yeast two-hybrid library in the Gal4 AD vector, which includes but is not limited to pACT2 as described in Hua et al., Construction of a modular yeast two-hybrid cDNA library from human EST clones for the human genome protein linkage map, Gene, Volume 215, Issue 1, 17 July 1998, Pages 143-152, the entire disclosure of which is incorporated by reference. Another complementary approach to address mammalian protein-protein interactions is to use a mammalian two-hybrid system as described in Luo et al., Mammalian Two-Hybrid System: A Complementary Approach to the Yeast Two-Hybrid System, BioTechniques 22:350-352, February 1997, incorporated in its entirety by reference herein. In a mammalian two-hybrid system, one protein of interest is expressed as a fusion to the Gal4 DNA binding domain and the other protein is expressed as a fusion to the activation domain of the herpes simplex virus VP16 protein. The vectors expressing these fusion proteins are cotransfected with a reporter chloramphenicol acetyltransferase (CAT) vector into a mammalian cell line. The cat gene contained in the reporter plasmid is under the control of five consensus Gal4 binding sites and if the two fusion proteins interact with one another there will be a significant increase in cat gene expression. The advantages of this system as that assay results may be obtained within 48 hrs of transfection and protein interactions in mammalian cells may be better indicative of in vivo protein interactions.

Furthermore since the overlap between human and yeast, C. elegans, or fly protein-protein interaction networks is limited (Gandhi et al. 2006), genetic interactions of the gene encoding the variant β₂M protein may be examined in another mammalian setting by utilizing methods that include but are not limited to Radiation hybrid (RH) panels (Goss and Harris 1975; Gyapay et al. 1996; McCarthy et al. 1997; Stewart et al. 1997; Watanabe et al. 1999; Olivier et al. 2001; Hitte et al. 2005). Generation of an RH panel is initiated with lethal irradiation of a donor cell line, whereby random breaks in its genome are induced. The donor cell has a selectable marker, which may be thymidine kinase. The fragmented DNA is rescued by fusing the donor cell to a non-irradiated host cell line which lacks the selectable marker. Growing the fused cells in selective medium, for example, HAT, ensures that only host cells incorporating the selectable marker plus a random sample of donor DNA will propagate. These techniques are further encompassed in Lin et al. A genome-wide map of human genetic interactions inferred from radiation hybrid genotypes, Genome Res. 2010. 20: 1122-1132, the entire disclosure of which is incorporated herein by reference.

Any identified modulators of variant β₂M gene activity identified in any genetic interaction screen may be further studied for effects on the formation of amyloid fibrils comprising the variant β₂M protein or variant β₂M aggregation. In the presence of a modulator, aggregation of variant β₂M protein is “altered” or “modulated”. The various forms of the term “alteration” or “modulation” are intended to encompass both inhibition of variant β₂M aggregation and promotion of variant β₂M aggregation. Aggregation of variant β₂M is “inhibited” in the presence of the modulator when there is a decrease in the amount and/or rate of variant β₂M aggregation as compared to the amount and/or rate of variant β₂M aggregation in the absence of the modulator. The various forms of the term “inhibition” are intended to include both complete and partial inhibition of variant β₂M aggregation.

An embodiment of the invention is to provide a high throughput drug screening system wherein the effects of compounds available from libraries on amyloid fibrils comprising variant β₂M are studied.

A further embodiment of the invention is to provide a high throughput drug screening system wherein inhibitors of variant β₂M amyloid fibril formation are identified.

A further embodiment of the invention provides therapeutic compositions of test compounds that are inhibitors of amyloid formation identified in said screens and methods of treatment using said therapeutic compositions.

Yet another embodiment of the invention provides methods of testing whether a compound or composition inhibits variant β₂M amyloid formation. These methods may comprise:
adding an isolated or recombinant or engineered variant β₂M polypeptide to a solution under essentially physiological conditions;
incubating the solution at 4-37° C. whereby variant β₂M amyloid fibrils are formed;
adding the compound or composition to the variant β₂M amyloid fibrils and
determining whether the compound or composition inhibits the formation of variant β2M amyloid fibrils.

The discovery and development of a new drug or therapeutic small molecules occurs via two main stages. An initial discovery stage aims to identify and optimize chemical lead structures among the numerous compounds synthesized to interact with a molecular target putatively involved in the pathophysiology of a human disease. A development stage then follows that assesses the pharmacokinetics, safety and efficacy properties of those drugs found to be potential candidate in humans. Recent advances in drug discovery include the synergistic development of two new technologies in biomedical research known as Combinatorial Chemistry (CC) and High Throughput Screening (HTS). CC, via computer-aided drug design and automated organic synthesis, allows thousands of compounds (a library) of systematic variants of a parent chemical structure to be produced in parallel. Pharmaceutical researchers can now create in a relatively short time millions of new compounds designed to target a specific cellular substrate such as receptors, enzymes, structural proteins and DNA, thus increasing the need for rapid and broadly applicable methods to screen these compounds. While it is important to screen compounds for the targets they were designed for, it is also important to be able to screen compounds for their unintended targets to anticipate potential side effects of selected candidate drugs and to find new uses for these substances if the side effect turns out to be a desired property. The development of HTS has been making it feasible, through automation and miniaturization techniques, to screen upwards of millions of drug candidates a year with robotic workstations running continuously 24 hours a day, 7 days a week. Billions of animal cells expressing the molecular target against which a library is made are grown in 96, 384, or 1536 micro-well plates and, via automated drug and liquid delivery and computerized read-out devices, are tested for a biological response to the drugs (U.S. Pat. No. 6,468,736).

Small molecule libraries that maybe be utilized in the practice of embodiments of the invention may be procured via a number of ways. A wide variety are available through commercial vendors- libraries which include natural products, U.S. Food and Drug Administration approved drugs, compounds of known biological activity, compounds that target specific proteins and others are already available for purchase. National Institutes like the National Institute of Health and various academic institutions also provide access to a number of libraries. A number of libraries and their sources have been further described in Gordon EJ, Small-molecule screening: it takes a village . . . ACS Chem Biol. 2007 Jan. 23;2(1):9-16, the entire disclosure of which is incorporated by reference herein.

The methodology of CC and HTS screening maybe applied to identify modulators of variant β₂M amyloid formation. Given the highly fibrillogenic property of the protein in essentially physiological conditions, amyloid fibrils may be generated in multi-well plates and a large scale screen run with compounds in different libraries to determine the effect of each compound on amyloid formation. Comparative analysis of amyloid structure both before and after the interaction with the compounds being tested may be carried out through methods discussed previously (eg. Congo red staining or light scattering). Should a particular test compound inhibit amyloid formation then it may be used in a therapeutic composition for the treatment of amyloidosis.

A test compound so identified using the methods of the invention or a pharmaceutically acceptable salt thereof is administered to a subject, patient or animal, typically in need of treatment, preferably a mammal, more preferably a human, suffering from amyloidosis. In one embodiment, “treatment” or “treating” refers to an amelioration of a disease, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease, either physically, e.g., stabilization of a discernible symptom, physiologically, e.g., stabilization of a physical parameter, or both. In yet another embodiment, “treatment” or “treating” refers to delaying the onset of a disease.

In certain embodiments, a test compound or a pharmaceutically acceptable salt thereof is administered to a subject, patient or animal, typically in need of treatment, preferably a mammal, more preferably a human, as a preventative measure against amyloidosis. As used herein, “prevention” or “preventing” refers to a reduction of the risk of acquiring a disease. In one embodiment, a test compound or a pharmaceutically acceptable salt thereof is administered as a preventative measure to a patient. According to this embodiment, the patient can have a genetic predisposition to a disease, such as a family history of the disease, or a non-genetic predisposition to the disease. Accordingly, the test compound and pharmaceutically acceptable salts thereof can be used for the treatment of one manifestation of a disease and prevention of another.

When administered to a patient, a test compound or a pharmaceutically acceptable salt thereof is preferably administered as component of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a test compound and pharmaceutically acceptable salts thereof.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a test compound or a pharmaceutically acceptable salt thereof into the bloodstream.

In specific embodiments, it may be desirable to administer a test compound or a pharmaceutically acceptable salt thereof locally. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce a test compound or a pharmaceutically acceptable salt thereof into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, a test compound and pharmaceutically acceptable salts thereof can be formulated as a suppository, with traditional binders and vehicles such as triglycerides.

In another embodiment, a test compound and pharmaceutically acceptable salts thereof can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, a test compound and pharmaceutically acceptable salts thereof can be delivered in a controlled release system (see, e.g., Goodson, in Medical Applications of Controlled Release, supra, vol. 2, pp. 115-138 (1984)). Other controlled-release systems discussed in the review by Langer, 1990, Science 249:1527-1533) may be used. In one embodiment, a pump may be used (see Langer, supra; Sefton, 1987, CRC Crit. Ref Biomed. Eng. 14:201; Buchwald et al., 1980, Surgery 88:507 Saudek et al., 1989, N. Engl. J. Med. 321:574). In another embodiment, polymeric materials can be used (see Medical Applications of

Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of a target RNA of a test compound or a pharmaceutically acceptable salt thereof, thus requiring only a fraction of the systemic dose.

Compositions comprising a test compound or a pharmaceutically acceptable salt thereof (“therapeutic compositions” or “test compound compositions”) can additionally comprise a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Test compound compositions, if desired, can also contain minor amounts of wetting or emulsifying agents, or pH buffering agents.

Test compound compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington's Pharmaceutical Sciences, Alfonso R. Gennaro ed., Mack Publishing Co. Easton, Pa., 19th ed., 1995, pp. 1447 to 1676, incorporated herein by reference.

In a preferred embodiment, a test compound or a pharmaceutically acceptable salt thereof is formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, and the like. Such vehicles are preferably of pharmaceutical grade. Typically, compositions for intravenous administration comprise sterile isotonic aqueous buffer. Where necessary, the compositions may also include a solubilizing agent.

In another embodiment, a test compound or a pharmaceutically acceptable salt thereof can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where a test compound or a pharmaceutically acceptable salt thereof is to be administered by infusion, it can be dispensed, for example, with an infusion bottle containing sterile pharmaceutical grade water or saline. Where the test compound or a pharmaceutically acceptable salt thereof is administered by injection, an ampoule of sterile water for injection or saline can be provided so that the ingredients may be mixed prior to administration.

The amount of a test compound or a pharmaceutically acceptable salt thereof that will be effective in the treatment of a particular disease will depend on the nature of the disease, and can be determined by standard clinical techniques. In addition, in vitro or in vivo assays may optionally be employed to help identify optimal dosage ranges. The precise dose to be employed will also depend on the route of administration, and the seriousness of the disease, and should be decided according to the judgment of the practitioner and each patient's circumstances. However, suitable dosage ranges for oral administration are generally about 0.001 milligram to about 200 milligrams of a test compound or a pharmaceutically acceptable salt thereof per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one test compound is administered, or if a test compound is administered with a therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

A test compound and pharmaceutically acceptable salts thereof are preferably assayed in vitro and in vivo, for the desired therapeutic or prophylactic activity, prior to use in humans. For example, in vitro assays can be used to determine whether it is preferable to administer a test compound, a pharmaceutically acceptable salt thereof, and/or another therapeutic agent. Animal model systems can be used to demonstrate safety and efficacy.

A variety of test compounds can be used for treating or preventing diseases in mammals. Types of test compounds include, but are not limited to, peptides, peptide analogs including peptides comprising non-natural amino acids, e.g., D-amino acids, phosphorous analogs of amino acids, such as .alpha.-amino phosphonic acids and .alpha.-amino phosphinic acids, or amino acids having non-peptide linkages, nucleic acids, nucleic acid analogs such as phosphorothioates or peptide nucleic acids (“PNAs”), hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose.

The entire disclosure of all the following patents and patent publications are herein incorporated by reference to aid in the practice of the claimed invention: U.S. Pat. Nos. and Publications Nos. 6,033,862; 7,691,897; 8,022,075; 7,658,917; 7,148,001; 6,468,736; 6,126,918; 6,503,713; 8,148,072; 8,137,666; 8,133,675; 8,133,674; 8,114,597; 8,110,358; 8,101,358; 8,067,173; 8,058,410; 8,026,070; 8,008,026; 7,867,727; 7,763,747; 7,662,558;7,598,031; 7,514,583; 7,488,605; 7,455,978; 6,727,070; 6,586,389; 6,498,017; 6,319,498, 5,958,883 and 20100249233.

Aspects of the invention will now be further described by way of the following non-limiting examples.

### EXAMPLE 1

Methodology: Informed consent and medical care are in accordance with the Declaration of Helsinki. Amyloid is detected by Congo red staining of sections of formalin fixed wax embedded biopsies. Immunohistochemical staining is carried out with monoclonal antibodies against serum amyloid A protein, κ and λ immunoglobulin light chains, transthyretin, fibrinogen, apolipoprotein AI, lysozyme and β₂M (Tennent G A, Cafferty K D, Pepys M B, Hawkins P N.

Congo red overlay immunohistochemistry aids classification of amyloid deposits. In: Kyle R A, Gertz M A, eds. Amyloid and Amyloidosis 1998. Pearl River, N.Y.: Parthenon Publishing; 1999:160-2). Polyclonal rabbit anti-β₂M antibody and gold-conjugated goat anti-rabbit IgG are used for immuno electron microscopy (Bridoux F, Sirac C, Hugue V, et al. Fanconi's syndrome induced by a monoclonal Vkappa3 light chain in Waldenstrom's macroglobulinemia. Am J Kidney Dis 2005;45:749-57). Amyloid fibrils are extracted (Tennent G A. Isolation and characterization of amyloid fibrils from tissue. In: Wetzel R, ed. Methods in Enzymology: Amyloid, Prions and Other Protein Aggregates. San Diego, Calif.: Academic Press Ltd; 1999:26-47) from the post mortem spleen of the proband's elder sister (case II.1) and additionally captured from cardiac and hepatic formalin fixed biopsy specimens by laser microdissection.

The amyloid proteome is analyzed by tandem mass spectrometry (Vrana J A, Gamez J D, Madden B J, Theis J D, Bergen H R, 3rd, Dogan A. Classification of amyloidosis by laser microdissection and mass spectrometry-based proteomic analysis in clinical biopsy specimens. Blood 2009;114:4957-9). The β₂M gene is sequenced in all available family members (Gussow D, Rein R, Ginjaar I, et al. The human beta 2-microglobulin gene. Primary structure and definition of the transcriptional unit. J Immunol 1987;139:3132-8). Mutation nomenclature is based on the β₂M transcript reference (NCBI RefSeq cDNA accession number NM_{—}004048).

The wild type β₂M mRNA molecule (SEQ ID NO: 1) of accession number NM_{—}004048 is represented below with the ATG start codon highlighted:

| | |
|---|---|
| 1 | aatataagtg gaggcgtcgc gctggcgggc attcctgaag ctgacagcat tcgggccgag |
| 61 | atgtctcgct ccgtggcctt agctgtgctc gcgctactct ctctttctgg cctggaggct |
| 121 | atccagcgta ctccaaagat tcaggtttac tcacgtcatc cagcagagaa tggaaagtca |
| 181 | aatttcctga attgctatgt gtctgggttt catccatccg acattgaagt tgacttactg |
| 241 | aagaatggag agagaattga aaaagtggag cattcagact tgtctttcag caaggactgg |
| 301 | tctttctatc tcttgtacta cactgaattc acccccactg aaaaagatga gtatgcctgc |
| 361 | cgtgtgaacc atgtgacttt gtcacagccc aagatagtta agtgggatcg agacatgtaa |
| 421 | gcagcatcat ggaggtttga agatgccgca tttggattgg atgaattcca aattctgctt |
| 481 | gcttgctttt taatattgat atgcttatac acttacactt tatgcacaaa atgtagggtt |
| 541 | ataataatgt taacatggac atgatcttct ttataattct actttgagtg ctgtctccat |
| 601 | gtttgatgta tctgagcagg ttgctccaca ggtagctcta ggagggctgg caacttagag |
| 661 | gtggggagca gagaattctc ttatccaaca tcaacatctt ggtcagattt gaactcttca |
| 721 | atctcttgca ctcaaagctt gttaagatag ttaagcgtgc ataagttaac ttccaattta |
| 781 | catactctgc ttagaatttg ggggaaaatt tagaaatata attgacagga ttattggaaa |
| 841 | tttgttataa tgaatgaaac attttgtcat ataagattca tatttacttc ttatacattt |
| 901 | gataaagtaa ggcatggttg tggttaatct ggtttatttt tgttccacaa gttaaataaa |
| 961 | tcataaaact tgatgtgtta tctctta |

The wild type β₂M gene translation product (SEQ ID NO: 2) is represented below including a 20 amino acid signal peptide (amino acids 1-20, underlined):

| | |
|---|---|
| 1 | M S R S V A L A V L A L L S L S G L E A I Q R T P K I Q V Y |
| 31 | S R H P A E N G K S N F L N C Y V S G F H P S D I E V D L L |
| 61 | K N G E R I E K V E H S D L S F S K D W S F Y L L Y Y T E F |
| 91 | T P T E K D E Y A C R V N H V T L S Q P K I V K W D R D M |

The 99 amino acid mature wild type β₂M protein transcript (SEQ ID NO: 3) excluding signal peptide is indicated below with the Aspartic acid, Asp (D) at position 76 highlighted:

| | |
|---|---|
| 1 | I Q R T P K I Q V Y S R H P A E N G K S N F L N C Y V S G F |
| 31 | H P S D I E V D L L K N G E R I E K V E H S D L S F S K D W |
| 61 | S F Y L L Y Y T E F T P T E K D E Y A C R V N H V T L S Q P |
| 91 | K I V K W D R D M |

The 99 amino acid mature mutant or variant β₂M protein transcript (SEQ ID NO: 4) is indicated below with the mutation of Aspartic acid, Asp to Asparagine, Asn (D76N). The Asp (N) at position 76 is highlighted:

| | |
|---|---|
| 1 | I Q R T P K I Q V Y S R H P A E N G K S N F L N C Y V S G F |
| 31 | H P S D I E V D L L K N G E R I E K V E H S D L S F S K D W |
| 61 | S F Y L L Y Y T E F T P T E K N E Y A C R V N H V T L S Q P |
| 91 | K I V K W D R D M |

Nucleotides are numbered according to the cDNA with +1 corresponding to the A of ATG translation initiation codon according to the guidelines available on the website of the Human Genome Variation Society. Variant protein nomenclature does not include the 20 amino acid signal peptide. Based on the provision of the variant β₂M protein amino acid sequence, the mRNA that encodes for the variant β₂M protein can be determined by one of ordinary skill in the art by identifying the codons and taking into account code degeneracy. cDNA and genomic DNA may be further isolated by methods known in the art.

Whole body anterior and posterior scintigraphic imaging is undertaken 24 hours after administration of ¹²³I labeled serum amyloid P component (SAP) (Hawkins P N, Lavender J P, Pepys M B. Evaluation of systemic amyloidosis by scintigraphy with ¹²³I-labeled serum amyloid P component. N Engl J Med 1990;323:508-13).

Recombinant wild type and variant β₂M proteins are expressed and purified (Esposito G, Ricagno S, Corazza A, et al. The controlling roles of Trp60 and Trp95 in beta2-microglobulin function, folding and amyloid aggregation properties. J Mol Biol 2008;378:887-97). The QuickChange™ site-directed mutagenesis kit and primer sequence

CACCCCCACTGAAAAAAATGAGTATGCCTGCC (SEQ ID NO: 5) are used for mutagenesis of Asp76 into Asn.

Gel bands of both recombinant and extracted fibril proteins are digested separately with AspN and with trypsin. AspN/trypsin sequential digestion confirms that the two isoforms are distinguishable by tryptic peptide analysis. AspN only cleaves if Asp is present at position 76; in the presence of Asn76 the uncleaved peptide, which is not released from the acrylamide gel, is cleaved by trypsin into peptides that are identifiable by mass spectrometry.

Protein stability was determined by fluorescence emission after incubation with guanidine hydrochloride (Esposito G, Ricagno S, Corazza A, et al. The controlling roles of Trp60 and Trp95 in beta2-microglobulin function, folding and amyloid aggregation properties. J Mol Biol 2008;378:887-97). Fibrillogenesis was studied in 40 μM β₂M at low pH (Naiki H, Hashimoto N, Suzuki S, Kimura H, Nakakuki K, Gejyo F. Establishment of a kinetic model of dialysis-related amyloid fibril extension in vitro. Amyloid-International Journal of Experimental and Clinical Investigation 1997;4:223-32) at neutral pH with 20% trifluoroethanol (Yamamoto S, Yamaguchi I, Hasegawa K, et al. Glycosaminoglycans enhance the trifluoroethanol-induced extension of beta 2-microglobulin-related amyloid fibrils at a neutral pH. J Am Soc Nephrol 2004;15:126-33) and in phosphate buffer at pH 7.4 with and without seeds of preformed β₂M fibrils and heparin (Jahn T R, Parker M J, Homans S W, Radford S E. Amyloid formation under physiological conditions proceeds via a native-like folding intermediate. Nat Struct Mol Biol 2006;13:195-201). Fibrils are characterized by transmission electron microscopy, cross polarized light microscopy, and ¹²⁵I SAP binding (Tennent G A. Isolation and characterization of amyloid fibrils from tissue. In: Wetzel R, ed. Methods in Enzymology: Amyloid, Prions and Other Protein Aggregates. San Diego, Calif.: Academic Press Ltd; 1999:26-47). The D76N variant is crystallised, X-ray diffraction data collected at 1.40 A resolution and the 3D structure solved (Esposito G, Ricagno S, Corazza A, et al. The controlling roles of Trp60 and Trp95 in beta2-microglobulin function, folding and amyloid aggregation properties. J Mol Biol 2008;378:887-97).

### EXAMPLE 2

### Experimental Procedures

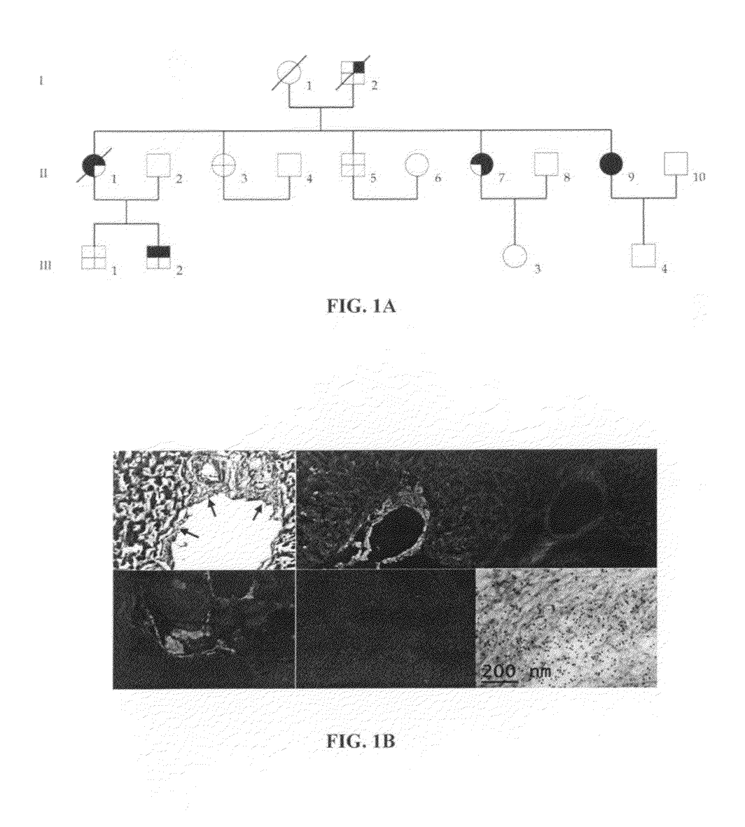

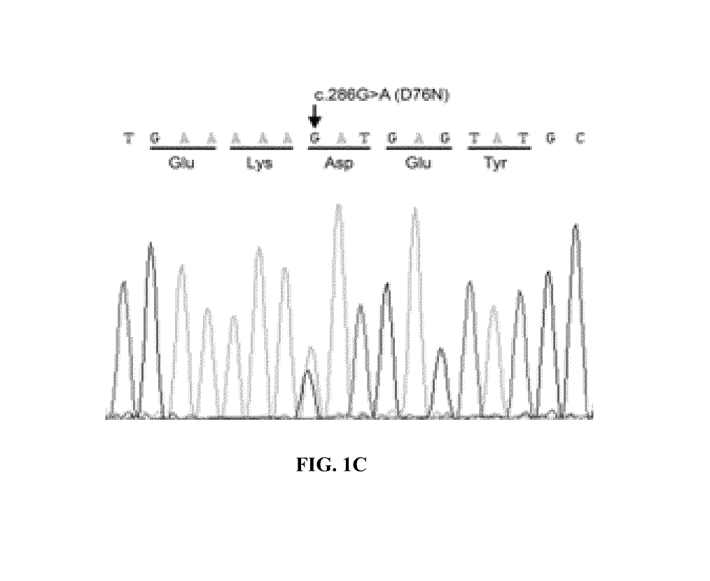

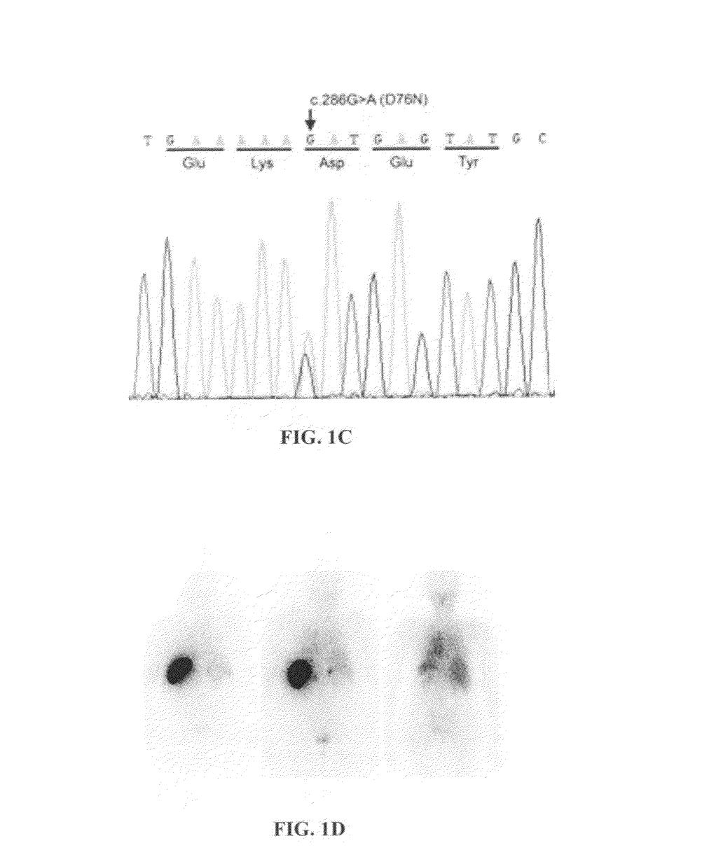

Patients: The proband (II.7) developed alternating diarrhea and constipation, and persistent sicca syndrome at age 62 years. The diarrhea gradually worsened and she became intermittently incontinent of faeces. She lost 20 kg of weight over 2 years and developed postural dizziness. She had no peripheral sensory or joint symptoms and no dyspnea or edema. The proband's elder sister (II.1) died at age 70 years following a progressive 20 year illness with initial diarrhea and weight loss, and persistent sicca syndrome followed by symmetrical sensorimotor axonal polyneuropathy and severe orthostatic hypotension. The proband's younger sister (II.9) was 56 years old and had suffered 6 years of chronic diarrhea, weight loss and sicca syndrome. Endoscopic bowel examination did not reveal a cause for the diarrhea in any case. Two further elder siblings (II.3 and II.5) were fit and well but the son of the proband's elder sister (III.2) had recently developed chronic diarrhea and sicca syndrome, aged 48 years (FIG. 1A). Serum biochemistry, including creatinine values in the range 63-72 gmol/L, was normal throughout the disease course in all 4 affected individuals. Severe autonomic neuropathy was confirmed in the proband, II.7, and her younger sister, II.9, by specialized functional tests.

Histology: The spleen, liver, heart, salivary glands and nerves of the proband's elder sister (II.1) contained extensive amyloid deposits at post mortem. Neural deposits are mainly around endoneurial capillaries and within vessel walls. Salivary gland and colonic biopsies from the proband's younger sister (II.9) also contained amyloid deposits. In both cases, antibodies against β₂M bound specifically to all the deposits, in light and electron microscopy studies, but there is no binding of antibodies against the other known amyloid fibril proteins (FIG. 1B). Proteomic analysis of amyloid deposits from the heart, spleen and liver of case II.1 confirmed that β₂M was the fibril protein.

β₂M Characterization: Each of the 4 clinically affected individuals, II.1, 11.7, 11.8 and 111.2, is heterozygous for a single base substitution c.286G>A (GAT/AAT) in the β₂M gene, encoding replacement of the negatively charged aspartic acid residue at position 76 of the mature protein by an uncharged asparagine (Asp76Asn) (FIG. 1C). No amyloidogenic mutations are identified in the genes for any other known amyloid fibril proteins, and the β₂M gene sequences of the three unaffected family members, II.3, 11.5, and III.1, are all wild type. The plasma β₂M concentration is 1.35 1.49 mg/L, within the normal reference range, throughout the disease course in both affected cases, II.7 and II.9, in whom it is measured.

SAP scintigraphy and cardiac magnetic resonance imaging: SAP scintigraphy in the proband and her symptomatic younger sister demonstrate a heavy visceral amyloid burden in both, involving especially the spleen and adrenal glands; neural and salivary gland deposits are not detected by routine SAP scintigraphy (FIG. 1D). Cardiac magnetic resonance imaging shows no signs of cardiac amyloidosis in either patient.

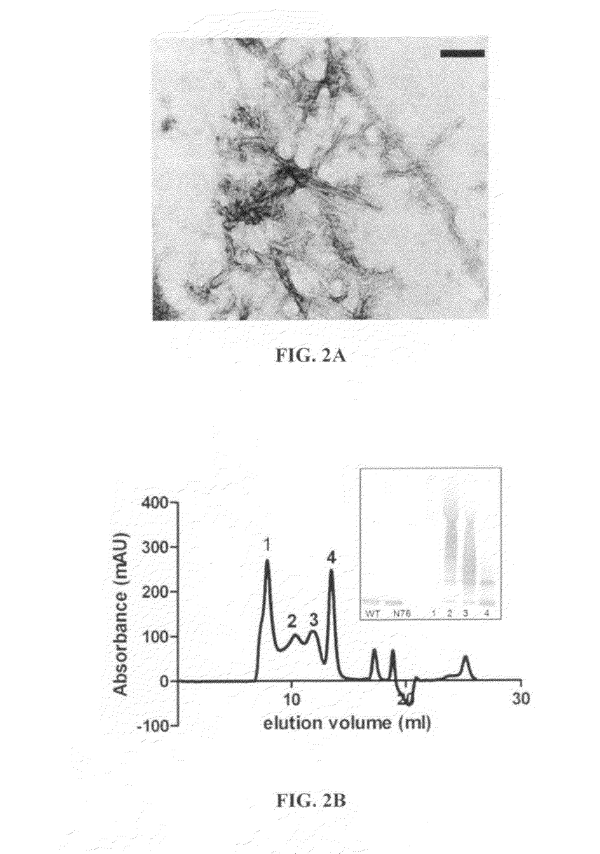

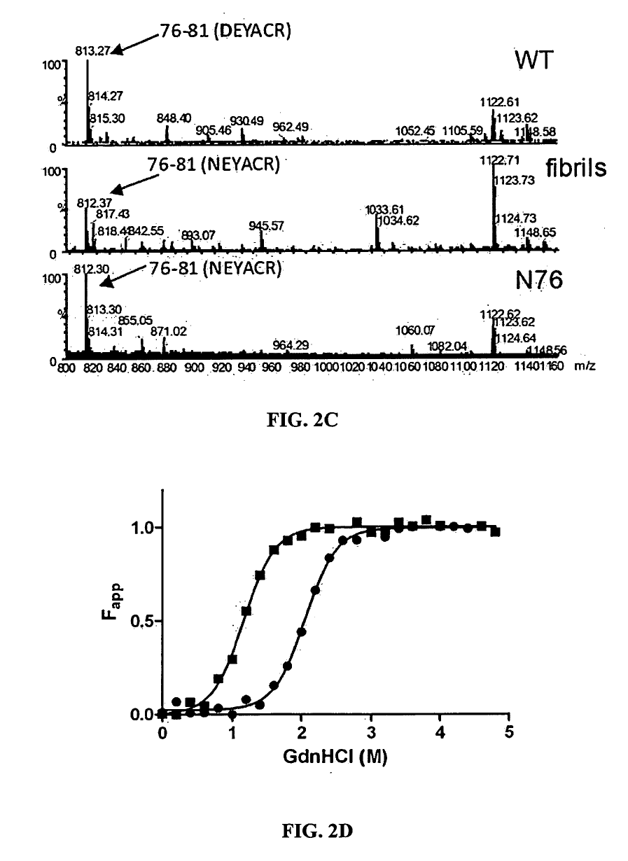

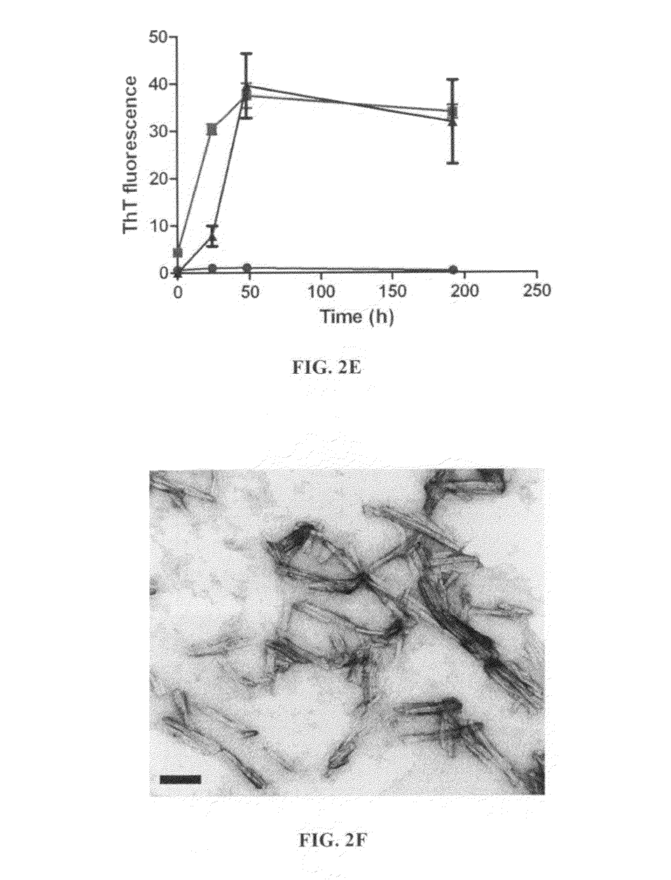

Characterization of ex-vivo amyloid fibrils: The β₂M isolated from extracted amyloid fibrils (FIG. 2A) comprises only full length variant protein (FIG. 2(B-C)). Furthermore, 2D gel electrophoresis, proteomic analysis and direct N terminal sequencing did not detect any β₂M with

N terminal truncation at residue 6, which is always present in dialysis related β₂M amyloid deposits (Giorgetti S, Stoppini M, Tennent G A, et al. Lysine 58-cleaved beta2-microglobulin is not detectable by 2D electrophoresis in ex vivo amyloid fibrils of two patients affected by dialysis-related amyloidosis. Protein Sci 2007;16:343-9) and which was recently hypothesized to be essential for formation of β₂M amyloid in vivo (Eichner T, Radford S E. Understanding the complex mechanisms of beta2-microglobulin amyloid assembly. Febs J 2011;278:3868-83).

Biochemical properties of D76N variant β₂M: Recombinant D76N β₂M is expressed and purified as a fully folded protein. The Asn for Asp76 substitution reduced β₂M stability by ˜2 kcal/mol, with Cₘ (±SD) falling from 2.0±0.2 M guanidine hydrochloride for wild type to 1.15±0.1 M for the variant (FIG. 2D). At low pH and in the presence of trifluoroethanol, wild type and D76N variant β32M formed fibrils at very similar rates. However, in physiologic solvent conditions the D76N variant was fully converted into fibrils with classical amyloid like properties within 48 hours whereas the wild type protein did not aggregate at all (FIG. 2(E-F)). Indeed, the variant aggregated at a similar rate in the absence of seeds and heparin, which are essential to promote fibrillogenesis by wild type β₂M. Fibril formation by the variant was markedly enhanced by shaking the protein solution, a recognized promoter of β sheet aggregation (Hoernke M, Falenski J A, Schwieger C, Koksch B, Brezesinski G. Triggers for beta-sheet formation at the hydrophobic-hydrophilic interface: high concentration, in-plane orientational order, and metal ion complexation. Langmuir: the ACS Journal of Surfaces and Colloids 2011;27:14218-31) which over-exposes the protein to the water-air interface mimicking the in vivo environment at the interface between polar and non-polar surfaces.

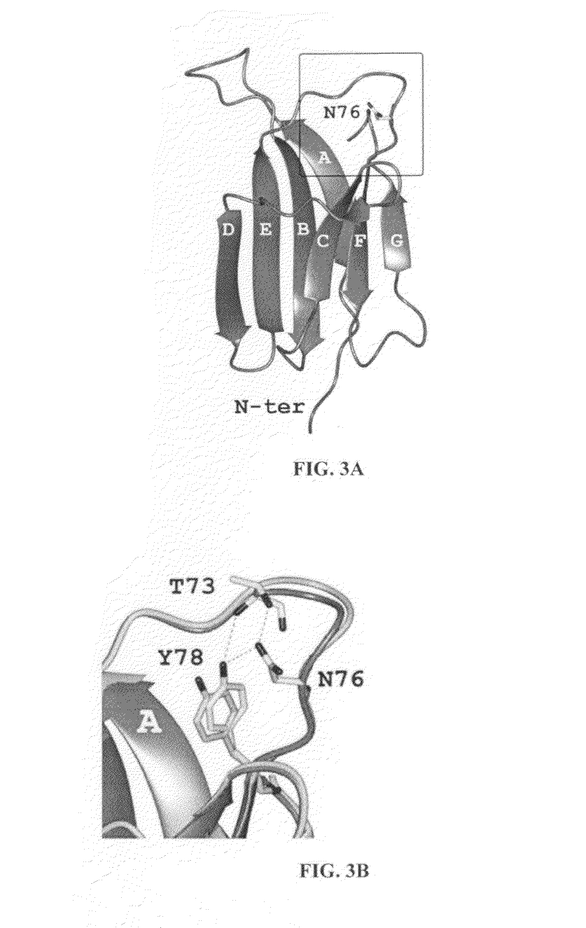

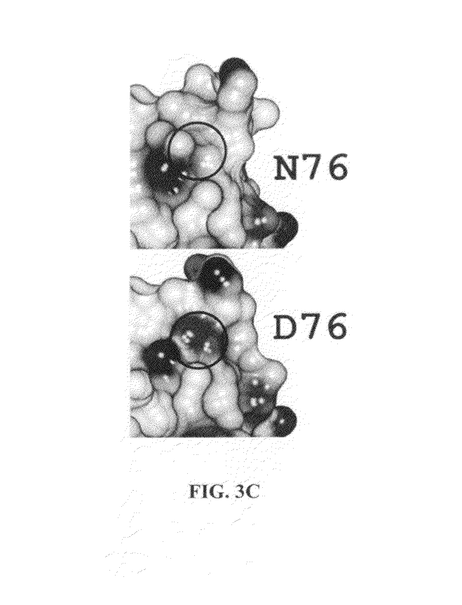

Crystal structure of D76N variant β₂M: The high resolution crystal structures of D76N variant 12M, at 1.40 Å, and wild type β₂M (PDB code 2YXF), matched closely, with a root mean square difference of 0.59 Å calculated over the whole Cα backbone (Esposito G, Ricagno S, Corazza A, et al. The controlling roles of Trp60 and Trp95 in beta2-microglobulin function, folding and amyloid aggregation properties. J Mol Biol 2008;378:887-97). The residue 76 Asn for Asp substitution has two notable effects. (1) The Asn76 amide establishes a new H-bond with Tyr78, which consequently moves about 1.5 Å closer to residue 76. In its new position, Tyr78 provides a hydrogen bond to the amide nitrogen of Thr73 (FIG. 3(A-C)). (2) The theoretical pI moves from 6.05 to 6.40, which is of particular interest since the negative wild type Asp76 residue partly balances the positive charges of the neighboring Lys41 and Lys75 residues, whereas this region of the protein becomes strongly electropositive in the D76N variant (FIG. 3C)). The crystallographic coordinates of the D76N variant β₂M protein is provided in TABLE 1 (filed on a CD).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An isolated or recombinant or engineered variant β₂-microglobulin (β₂M) polypeptide, wherein the polypeptide is amyloidogenic under essentially physiological conditions in vitro.

2. The polypeptide according to claim 1, wherein the polypeptide is amyloidogenic when added to a solution comprising 25 mM phosphate buffer at pH 7.4 at 4-37° C.

3. The polypeptide according to claim 1, wherein the polypeptide is amyloidogenic in a normal saline solution.

4. The polypeptide according to claim 1, wherein the polypeptide has a substitution at amino acid position 76 with reference to the position numbering of SEQ ID NO: 3.

5. The polypeptide according to claim 4, wherein the substitution is D76N.

6. The polypeptide according to claim 1, wherein the polypeptide comprises the amino acid sequence of SEQ ID NO: 4.

7. A polypeptide having at least 95% identity with the polypeptide of claim 1.

8. A polypeptide having at least 95% identity with the polypeptide of claim 4.

9. A polypeptide having at least 95% identity with the polypeptide of claim 6.

10. A nucleic acid molecule encoding the polypeptide of claim 1.

11. A nucleic acid molecule encoding the polypeptide of claim 4.

12. A nucleic acid molecule encoding the polypeptide of claim 6.

13. A recombinant vector expressing the nucleic acid molecules of anyone of claim 10, 11 or 12.

14. The vector of claim 13, wherein the vector is a plasmid.

15. The plasmid of claim 14, wherein the plasmid is pHN1.

16. A host cell expressing the vector of claim 13.

17. A host cell expressing the plasmid of claim 14.

18. A composition comprising the polypeptide of claim 1.

19. The composition of claim 18, wherein the composition further comprises an aggregation enhancing agent.

20. The composition of claim 19, wherein the aggregation enhancing agent is selected from the group consisting of a cation, a glycosaminoglycan, a lysophosphatidic acid, a non-esterified fatty acid or a collagen.

21. The composition of claim 19, wherein the cation is Cu²⁺.

22. A method of forming variant β₂M amyloid fibrils in vitro, wherein the method comprises:
adding an isolated or recombinant or engineered variant β₂M polypeptide to a solution under essentially physiological conditions, wherein the polypeptide has a substitution at amino acid position 76 with reference to the position numbering of SEQ ID NO: 3;
incubating the solution at 37° C.; whereby variant β₂M amyloid fibrils are formed; and
determining that the variant β₂M amyloid fibrils formed specifically bind Congo red from an alkaline alcoholic solution and then show red/green birefringence when viewed under crossed polarized light.

23. The method according to claim 22, wherein the solution comprises a phosphate buffer.

24. The method according to claim 22, wherein the solution comprises a normal saline solution.

25. The method according to claim 22, wherein the solution comprises heparin.

26. The method according to claim 25, wherein concentration of the heparin is 100 μg/ml.

27. The method according to claim 22, wherein the solution comprises wild type β₂M fibril seeds.

28. The method according to claim 22, wherein concentration of the wild type β₂M fibril seeds is 25 μg/ml.

29. The method according to claim 22, wherein the solution is agitated.

30. The method according to claim 29, wherein the agitation solution is agitated at 225 rpm.

31. A method of testing whether a compound or composition inhibits variant β₂M amyloid formation comprising:
adding an isolated or recombinant or engineered variant β₂M polypeptide to a solution under essentially physiological conditions;
incubating the solution at 4-37° C. whereby variant β₂M amyloid fibrils are formed;
adding the compound or composition to the variant β₂M amyloid fibrils, and
determining whether the compound or composition inhibits the formation of variant β₂M amyloid fibrils.

32. The polypeptide of claim 1 having a crystal structure having the coordinates listed in TABLE 1.

33. The method according to claim 22, wherein the substitution is D76N.
